(19) United States
(12) Patent Application Publication
(10) Pub. No.: US 2026/0217783 A1
(43) Pub. Date: Jul. 30, 2026

(54) METHODS OF REDUCING CONTAMINANTS IN PROTEIN PURIFICATION

(71) Applicant: ELI LILLY AND COMPANY, Indianapolis, IN (US)

(72) Inventors: Amy Lynn HUEBNER, Bridgewater, NJ (US); Timothy BLANC, Fair Haven, NJ (US)

(21) Appl. No.: 19/141,221

(22) PCT Filed: Dec. 19, 2023
(86) PCT No.: PCT/US2023/084942

§ 371 Date: Jun. 19, 2025

Related U.S. Application Data
(60) Provisional application No. 63/476,040, filed on Dec. 19, 2022.

(51) Int. Cl. C07K 14/605; G01N 33/543; G01N 33/68

(52) U.S. Cl. CPC C07K 14/605; G01N 33/54386; G01N 33/6848; C07K 2319/30; G01N 2474/00

(57) ABSTRACT

Provided herein are improved methods of purifying an Fc-containing protein from a mixture comprising the Fc-containing protein and one or more host cell protein (HCP). The methods generally involve applying the mixture to a chromatography column comprising a protein A chromatography matrix, and washing the chromatography matrix with a sodium caprylate wash buffer.

### BACKGROUND

Production of recombinant Fc-containing proteins for therapeutic use typically involves expression of the proteins in mammalian cells and subsequent purification of these proteins from host cell contaminants. Protein A affinity chromatography is commonly used as part of the purification process for Fc-containing proteins, because of the ability of protein A to selectively bind to the Fc region of the Fc-containing proteins. Although protein A affinity chromatography removes a large amount of host cell contaminants, a significant amount of host cell proteins (HCPs) can still be present after such chromatography, including proteases, such as cathepsin D. These proteases can clip or fragment Fc-containing proteins, and lead to reduced yield of intact protein.

Accordingly, there is a need for improved methods of purifying Fc-containing proteins to remove HCPs, such as proteases (e.g., cathepsin D), using protein A affinity chromatography.

### SUMMARY

The present disclosure provides improved methods for purifying an Fc-containing protein (e.g., dulaglutide) from a mixture comprising the Fc-containing protein and one or more host cell protein (HCP). The methods generally involve applying the mixture to a chromatography column comprising a protein A chromatography matrix, under conditions whereby the dulaglutide binds to the chromatography matrix; and washing the chromatography matrix with a sodium caprylate wash buffer (e.g., a sodium caprylate wash buffer comprising 150-500 mM sodium caprylate). The methods disclosed herein are particularly advantageous in that they significantly reduce the levels of cathepsin D in the protein A chromatography column eluate, and significantly increase the amount of intact Fc-containing protein recovered during purification, compared to other wash methods known in the art.

In an aspect, provided herein is a method of purifying dulaglutide from a mixture comprising the dulaglutide and one or more host cell protein (HCP), the method comprising the steps of: (a) applying the mixture to a chromatography column comprising a Protein A chromatography matrix, under conditions whereby the dulaglutide binds to the chromatography matrix; and (b) washing the chromatography matrix with a sodium caprylate wash buffer comprising 150-500 mM sodium caprylate.

In an embodiment, the sodium caprylate wash buffer comprises 150-350 mM sodium caprylate. In an embodiment, the sodium caprylate wash buffer comprises 200-350 mM sodium caprylate. In an embodiment, the sodium caprylate wash buffer comprises about 300 mM sodium caprylate. In an embodiment, the sodium caprylate wash buffer comprises about 50 mM Tris. In an embodiment, the sodium caprylate wash buffer comprises 0.5-1.5 M NaCl. In an embodiment, the sodium caprylate wash buffer comprises about 1M NaCl. In an embodiment, the sodium caprylate wash buffer has a pH of 7-9. In an embodiment, the sodium caprylate wash buffer has a pH of about 8.

In an embodiment, the method further comprises washing the chromatography matrix with a NaCl wash buffer comprising 0.5-1.5 M NaCl. In an embodiment, the NaCl wash buffer comprises about 1M NaCl. In an embodiment, the NaCl wash buffer comprises about 50 mM Tris. In an embodiment, the NaCl wash buffer has a pH of 7-9. In an embodiment, the NaCl wash buffer has a pH of about 8.

In an embodiment, the chromatography column is washed with the NaCl wash buffer prior to being washed with the sodium caprylate wash buffer. In an embodiment, the chromatography column is washed with the sodium caprylate wash buffer prior to being washed with the NaCl wash buffer.

In an embodiment, the chromatography matrix is equilibrated with a Tris buffer at a pH of 7-9 between step (a) and step (b). In an embodiment, the Tris buffer comprises 10-100 mM Tris. In an embodiment, the Tris buffer comprises about 50 mM Tris. In an embodiment, the Tris buffer has a pH of about 8.0.

In an embodiment, the chromatography matrix is washed with 2-10 column volumes of the sodium caprylate wash buffer. In an embodiment, the chromatography matrix is washed with about 2 column volumes of the sodium caprylate wash buffer. In an embodiment, the chromatography matrix is washed with 2-10 column volumes of the NaCl wash buffer. In an embodiment, the chromatography matrix is washed with about 5 column volumes of the NaCl wash buffer.

In an embodiment, the chromatography matrix is sequentially washed with: about 5 column volumes of a NaCl wash buffer comprising about 50 mM Tris and about 1 M NaCl, with a pH of about 8; and about 2 column volumes of a sodium caprylate wash buffer comprising about 50 mM Tris and about 300 mM sodium caprylate, with a pH of about 8.

In an embodiment, the chromatography matrix is washed with the NaCl wash buffer at a flow rate of 300±30 cm/hr. In an embodiment, the chromatography matrix is washed with the sodium caprylate wash buffer at a flow rate of 300±30 cm/hr.

In an embodiment, the method further comprises washing the chromatography matrix with a Tris buffer comprising 10-100 mM Tris, optionally the Tris buffer comprises about 50 mM Tris at a pH of 8.0.

In an embodiment, the method further comprises contacting the chromatography matrix with an elution buffer to produce an eluate containing dulaglutide. In an embodiment, the elution buffer has a pH of 2.5-4. In an embodiment, the elution buffer has a pH of about 3. In an embodiment, the elution buffer comprises sodium citrate. In an embodiment, the elution buffer comprises 5-25 mM sodium citrate. In an embodiment, the elution buffer comprises about 10 mM sodium citrate.

In an embodiment, the eluate is monitored by absorbance using a spectrophotometer. In an embodiment, the eluate comprises at least about 70% of the dulaglutide present in the mixture prior to performing the method.

In an embodiment, the chromatography column has a diameter of 50-150 cm. In an embodiment, the chromatography column has a diameter of 75-150 cm. In an embodiment, the chromatography column has a diameter of about 100 cm. In an embodiment, the Protein A chromatography matrix has an average particle size of 80-90 μm. In an embodiment, the Protein A chromatography matrix has an average particle size of about 85 μm. In an embodiment, the Protein A chromatography matrix has an average particle size of 50-70 μm. In an embodiment, the Protein A chromatography matrix has an average particle size of about 60 μm.

In an embodiment, the protein A chromatography matrix is stable in alkali.

In an embodiment, the one or more HCP is a protease selected from the group consisting of serine protease, aspartic protease, cysteine protease, metalloprotease, and aminopeptidase, or a combination thereof. In an embodiment, the one or more HCP is cathepsin D.

In an embodiment, the eluate comprises a reduced amount of the one or more HCP compared to the mixture. In an embodiment, the eluate comprises no more than 100 ng/mg of HCPs. In an embodiment, the amount of one or more HCP is measured by mass spectrometry or ELISA.

In an embodiment, the eluate comprises a reduced amount of cathepsin D compared to the mixture. In an embodiment, the eluate comprises no more than 100 ng/mg cathepsin D. In an embodiment, the amount of cathepsin D is measured by mass spectrometry or ELISA. In an embodiment, the amount of cathepsin D is measured by an enzymatic assay. In an embodiment, the amount of cathepsin D is measured by an enzymatic assay wherein a fluorescent substrate of Cathepsin D is added to the sample and its cleavage product is measure by HPLC with Fluorescent detection.

In an embodiment, the mass spectrometry is LC-MS.

In an embodiment, the eluate comprises less than 300 U/mL cathepsin D activity.

In an aspect, provided herein is dulaglutide produced by any one of the methods disclosed herein.

### Additional Embodiments of the Present Disclosure are Described Below:

1. A method of purifying an Fc-containing protein from a mixture comprising the Fc-containing protein and one or more host cell protein (HCP), the method comprising the steps of:
(a) applying the mixture to a chromatography column comprising a Protein A chromatography matrix, under conditions whereby the Fc-containing protein binds to the chromatography matrix; and
(b) washing the chromatography matrix with a sodium caprylate wash buffer comprising 150-500 mM sodium caprylate.
   2. The method of embodiment 1, wherein the sodium caprylate wash buffer comprises 200-500 mM or 150-350 mM sodium caprylate.
   3. The method of embodiment 1 or 2, wherein the sodium caprylate wash buffer comprises 200-350 mM sodium caprylate.
   4. The method of any one of embodiments 1-3, wherein the sodium caprylate wash buffer comprises about 300 mM sodium caprylate.
   5. The method of any one of embodiments 1-4, wherein the sodium caprylate wash buffer comprises about 50 mM Tris and about 300 mM sodium caprylate.
   6. The method of any one of embodiments 1-5, wherein the sodium caprylate wash buffer comprises 0.5-1.5 M NaCl.
   7. The method of any one of embodiments 1-6, wherein the sodium caprylate wash buffer comprises about 1M NaCl.
   8. The method of any one of embodiments 1-7, wherein the sodium caprylate wash buffer has a pH of 7-9.
   9. The method of any one of embodiments 1-8, wherein the sodium caprylate wash buffer has a pH of about 8.
   10. The method of embodiment 1, further comprising washing the chromatography matrix with a NaCl wash buffer comprising 0.5-1.5 M NaCl.
   11. The method of embodiment 10, wherein the NaCl wash buffer comprises about 1M NaCl.
   12. The method of embodiment 10 or 11, wherein the NaCl wash buffer comprises about 50 mM Tris and about 1M NaCl.
   13. The method of any one of embodiments 10-12, wherein the NaCl wash buffer has a pH of 7-9.
   14. The method of any one of embodiments 10-13, wherein the NaCl wash buffer has a pH of about 8.
   15. The method of any one of embodiments 10-14, wherein the chromatography column is washed with the NaCl wash buffer prior to being washed with sodium caprylate wash buffer.
   16. The method of any one of embodiments 10-14, wherein the chromatography column is washed with the sodium caprylate wash buffer prior to being washed with NaCl wash buffer.
   17. The method of any one of embodiments 1-16, wherein the chromatography matrix is equilibrated with a Tris buffer at a pH of 7-9 between step (a) and step (b).
   18. The method of embodiment 17, wherein the Tris buffer comprises 10-100 mM Tris.
   19. The method of embodiment 17 or 18, wherein the Tris buffer comprises about 50 mM Tris.
   20. The method of any one of embodiments 17-19, wherein the Tris buffer has a pH of about 8.0.
   21. The method of any one of embodiments 1-20, wherein the chromatography matrix is washed with 2-10 column volumes of the sodium caprylate wash buffer.
   22. The method of any one of embodiments 1-21, wherein the chromatography matrix is washed with about 2 column volumes of the sodium caprylate wash buffer.
   23. The method of any one of embodiments 10-22, wherein the chromatography matrix is washed with 2-10 column volumes of the NaCl wash buffer.
   24. The method of any one of embodiments 10-23, wherein the chromatography matrix is washed with about 2 or about 5 column volumes of the NaCl wash buffer.
   25. The method of embodiment 1, wherein the chromatography matrix is sequentially washed with:
(a) about 2 column volumes of a NaCl wash buffer comprising about 50 mM Tris and about 1 M NaCl, with a pH of about 8; and
(b) about 2 column volumes of a sodium caprylate wash buffer comprising about 50 mM Tris and about 300 mM sodium caprylate, with a pH of about 8.
   26. The method of any one of embodiments 10-25, wherein the chromatography matrix is washed with the NaCl wash buffer at a flow rate of 300=30 cm/hr.
   27. The method of any one of embodiments 1-26, wherein the chromatography matrix is washed with the sodium caprylate wash buffer at a flow rate of 300±30 cm/hr.
   28. The method of any one of embodiments 1-27, further comprising washing the chromatography matrix with a Tris buffer comprising 10-100 mM Tris, optionally wherein the Tris buffer comprises about 50 mM Tris at a pH of about 8.0.
   29. The method of any one of embodiments 1-28, further comprising contacting the chromatography matrix with an elution buffer to produce an eluate containing the Fc-containing protein.
   30. The method of embodiment 29, wherein the elution buffer has a pH of 2.5-4.
   31. The method of embodiment 29 or 30, wherein the elution buffer has a pH of about 3.
   32. The method of any one of embodiments 29-31, wherein the elution buffer comprises sodium citrate.
   33. The method of any one of embodiments 29-32, wherein the elution buffer comprises 5-25 mM sodium citrate.
   34. The method of any one of embodiments 29-33, wherein the elution buffer comprises about 10 mM sodium citrate.
   35. The method of any one of embodiments 29-34, wherein the eluate is monitored by absorbance using a spectrophotometer.
   36. The method of any one of embodiments 29-35, wherein the eluate comprises at least 70% of the Fc-containing protein present in the mixture.
   37. The method of any one of embodiments 1-36, wherein the chromatography column has a diameter of 75-150 cm.
   38. The method of any one of embodiments 1-37, wherein the chromatography column has a diameter of about 100 cm.
   39. The method of any one of embodiments 1-38, wherein the Protein A chromatography matrix has an average particle size of 80-90 μm.
   40. The method of any one of embodiments 1-39, wherein the Protein A chromatography matrix has an average particle size of about 85 μm.
   41. The method of any one of embodiments 1-40, wherein the protein A chromatography matrix is stable in alkali.
   42. The method of any one of embodiments 1-41, wherein the one or more HCP is a protease selected from the group consisting of serine protease, aspartic protease, cysteine protease, metalloprotease, and aminopeptidase, or a combination thereof.
   43. The method of embodiment 1-42, wherein the one or more HCP is cathepsin D.
   44. The method of any one of embodiments 29-43, wherein the eluate comprises a reduced amount of the one or more HCP compared to the mixture.
   45. The method of any one of embodiments 29-44, wherein the eluate comprises no more than 100 ng/mg of HCPs.
   46. The method of embodiment 43 or 44, wherein the one or more HCP is measured by mass spectrometry or ELISA.
   47. The method of any one of embodiments 29-46, wherein the eluate comprises a reduced amount of cathepsin D compared to the mixture.
   48. The method of any one of embodiments 29-47, wherein the eluate comprises no more than 100 ng/mg cathepsin D.
   49. The method of embodiment 47 or 48, wherein the amount of cathepsin D is measured by mass spectrometry or ELISA.
   50. The method of embodiment 46 or 49, wherein the mass spectrometry is LC-MS.
   51. The method of any one of embodiments 29-50, wherein the eluate comprises less than 300 μU/mL cathepsin D activity.
   52. The method of any one of embodiments 1-51, wherein the Fc-containing protein is not an antibody.
   53. The method of any one of embodiments 1-52, wherein the Fc-containing protein comprises a glucagon-like peptide 1 (GLP-1) analog comprising one or more modifications compared to a wild type GLP-1 amino acid sequence (SEQ ID NO: 1).
   54. The method of any one of embodiments 1-53, wherein the Fc-containing protein comprises a GLP-1 analog comprising the amino acid sequence of SEQ ID NO: 2.
   55. The method of any one of embodiments 1-54, wherein the Fc-containing protein comprises a peptide linker.
   56. The method of embodiment 55, wherein the peptide linker comprises 1-10 G₄S units (SEQ ID NO: 3).
   57. The method of any one of embodiments 1-56, wherein the Fc-containing protein comprises:
(a) a GLP-1 analog comprising the amino acid sequence of SEQ ID NO: 2;
(b) a peptide linker comprising the amino acid sequence of SEQ ID NO: 3; and
c) an Fc portion of an immunoglobulin.
   58. The method of embodiment 57, wherein the N-terminal residue of the peptide linker is directly fused to the C-terminal residue of the GLP-1 analog, and the C-terminal residue of the peptide linker is directly fused to the N-terminal residue of the Fc portion.
   59. The method of any one of embodiments 1-58, wherein the Fc-containing protein comprises the amino acid sequence of SEQ ID NO: 4.
   60. The method of any one of embodiments 1-59, wherein the wherein the Fc-containing protein is a homodimer comprising two identical amino acid chains each comprising the amino acid sequence of SEQ ID NO: 4.
   61. The method of any one of embodiments 1-60, wherein the Fc-containing protein is dulaglutide.
   62. An Fc-containing protein produced by the method of any one of the preceding claims.

### BRIEF DESCRIPTION OF DRAWINGS

FIG. 1 is a UV chromatogram of flow throughs from washes with the indicated buffer solutions on a Protein A chromatography column. The Protein A column was pre-loaded with a mixture comprising dulaglutide and host cell proteins prior to the wash steps.

FIG. 2 is a graph showing the HCP content in ng/mg as assessed by ELISA, the HCP content in ppm per unit of dulaglutide as assessed by LC-MS, and the cathepsin D activity in μU/mL in dulaglutide eluates after the dulaglutide mixtures were pre-loaded onto a Protein A column and washed with a 50 mM Tris, pH 8.0 buffer comprising the indicated concentrations of sodium caprylate. The wash conditions were 5 CV of 50 mM Tris, pH 8.0, then 5 CV of 50 mM Tris, 1M NaCl, pH 8, then 5 CV of 50 mM Tris, X mM (300, 200, 100, 50 mM, as indicated in the figure) sodium caprylate, pH 8, followed by 5 CV 50 mM Tris, pH 8.0.

FIG. 3 is a graph showing the HCP content in ng/mg as assessed by ELISA, the HCP content in ppm per unit of dulaglutide as assessed by LC-MS, and the cathepsin D activity in μU/mL in dulaglutide eluates after dulaglutide mixtures were pre-loaded onto a Protein A column and washed with the indicated combination or serial washes with an NaCl wash buffer and a sodium caprylate wash buffer. The first wash was the same for all test conditions, comprising five column volumes of 50 mM Tris, pH 8.0.

FIG. 4 is a graph showing the HCP content in ng/mg as assessed by ELISA, the HCP content in ppm per unit of dulaglutide as assessed by LC-MS, and the cathepsin D activity in μU/mL in dulaglutide eluates after dulaglutide mixtures were pre-loaded onto a Protein A column and followed by two washes: a first wash with five column volumes (CVs) of 50 mM Tris, pH 8.0, and a second wash with five CVs of a wash buffer containing 1 M NaCl as a control or the indicated CVs of a wash buffer containing 300 mM sodium caprylate. The control conditions were 5 CV of 50 mM Tris, pH 8.0, then 5 CV of 50 mM Tris, 1M NaCl, pH 8, then 5 CV of 50 mM Tris, pH 8.0; the 5 CV wash, 3 CV wash, and 2 CV wash conditions were 5 CV of 50 mM Tris, pH 8.0, then X CV (5, 3, 2 CV, as indicated in the figure) of 50 mM Tris, 1M NaCl, pH 8, then X CV of 50 mM Tris, 300 mM sodium caprylate, pH 8, then X CV 50 mM Tris, pH 8.0.

### DETAILED DESCRIPTION

Provided herein are improved methods for purifying an Fc-containing protein (e.g., dulaglutide) from a mixture comprising the Fc-containing protein and one or more host cell protein (HCP). The methods generally involve applying the mixture to a chromatography column comprising a protein A chromatography matrix, under conditions whereby the dulaglutide binds to the chromatography matrix; and washing the chromatography matrix with a sodium caprylate wash buffer (e.g., a sodium caprylate wash buffer comprising 150-500 mM sodium caprylate). The methods disclosed herein are particularly advantageous in that they significantly reduce the levels of cathepsin D in the protein A chromatography column eluate, and significantly increase the amount of intact Fc-containing protein recovered during purification, compared to other wash methods known in the art.

### I. Definitions

As used herein, the term “Fc-containing protein” refers to a protein comprising an Fc region. In an embodiment, the Fc-containing protein comprises a variant Fc region comprising one or more amino acid substitutions, additions, and/or deletions relative to a naturally occurring Fc region. In an embodiment, the Fc-containing protein is not an antibody.

As used herein, the term “contaminant” refers to any material, particularly a biological macromolecule such as DNA, RNA, or a protein, other than a recombinantly produced Fc-containing protein that is present in a mixture. Contaminants include, without limitation, cellular and viral proteins or nucleic acids, or byproducts thereof, that arise in the production process of an Fc-containing protein. A contaminant also includes any host cell protein (HCP), host cell nucleic acid, or host cell fragment that results from any stage of an Fc-containing protein production process.

The terms “host cell protein,” and “HCP,” are used herein to refer to any unwanted protein that originates from a cell (e.g., a mammalian cell) used to produce an Fc-containing protein.

As used herein, the term “purifying,” “purify,” or “purification” refers to reduction in the amount of a contaminant (e.g., an HCP) in a composition comprising an Fc-containing protein. Purification may or may not result in the complete removal of contaminants from a composition. In certain embodiments, purification refers to at least a 2-fold, 3-fold, 4-fold, 5-fold, 10-fold, 15-fold, 20-fold, 25-fold, 30-fold, 35-fold, 40-fold, 45-fold, or 50-fold reduction in contaminants.

As used herein, the term “eluate” refers to a solution comprising an Fc-containing protein (e.g., dulaglutide) that has been purified according to a method disclosed herein. In an embodiment, the eluate may also comprise one or more HCP. In an embodiment, the eluate comprises a lower amount of the HCP than a mixture.

As used herein, the term “antibody” includes full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above.

As used herein, the term “about,” when in reference to a value or parameter herein, includes a variability of +5% of the value or parameter. For example, when referring to a pH value, “about” refers to a range that includes the value 5% below the referenced value, and the value 5% above the referenced value. Thus, a pH of about 10 refers to a pH that encompasses a pH of 9.5 to a pH of 10.5, inclusive.

### II. Protein A Chromatography Methods

A challenge in the downstream processing of Fc-containing proteins (e.g., dulaglutide) is the efficient separation of the Fc-containing protein from contaminants and impurities, such as host cell proteins (HCPs). In particular, in the manufacturing process of dulaglutide, residual cathepsin D can clip the dulaglutide product and lead to a reduced yield of intact dulaglutide. The methods disclosed herein significantly reduce the level of HCPs, including cathepsin D, in the protein A chromatography column eluate, and significantly increase the amount of intact Fc-containing protein recovered during purification

In an aspect, provided herein is a method of purifying an Fc-containing protein from a mixture comprising the Fc-containing protein and one or more host cell protein (HCP), the method comprising the steps of: applying the mixture to a chromatography column comprising a Protein A chromatography matrix, under conditions whereby the Fc-containing protein binds to the chromatography matrix; and washing the chromatography matrix with a sodium caprylate wash buffer comprising 150-500 mM sodium caprylate.

In general, protein A chromatography method comprises the following steps in sequential order: preparation of the load composition comprising a mixture of the Fc-containing protein and one or more HCP, application of the load composition to the chromatography column comprising the protein A chromatography matrix, washing of the protein A chromatography matrix, and elution of the Fc-containing protein. The skilled artisan will appreciate that depending on the desired purpose and outcome, a protein A chromatography method can comprise additional intermediate steps, and/or additional steps before and after the protein A purification process.

In an aspect, provided herein is a method of purifying an Fc-containing protein from a mixture of the Fc-containing protein and one or more contaminant, the method comprising the steps of: applying the mixture to a chromatography column comprising a Protein A chromatography matrix, under conditions whereby the Fc-containing protein binds to the chromatography matrix; and washing the chromatography matrix with: a sodium caprylate wash buffer comprising 150-500 mM sodium caprylate.

In an embodiment, the methods further comprise washing the protein A chromatography matrix with an NaCl wash buffer comprising 0.5-1.5 M NaCl.

In an embodiment, the chromatography column is washed with the NaCl wash buffer before or after the chromatography matrix is washed with the sodium caprylate wash buffer. In an embodiment, the NaCl and sodium caprylate wash buffers are combined. In an embodiment, the chromatography matrix is washed with the NaCl wash buffer is followed by the wash with the sodium caprylate wash buffer.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; and washing the chromatography matrix with an NaCl buffer comprising 0.5-1.5 M NaCl, followed by washing the chromatography matrix with a sodium caprylate wash buffer comprising 150-500 mM sodium caprylate.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; and washing the chromatography matrix with about 2 column volumes of an NaCl buffer comprising about 1 M NaCl, followed by washing the chromatography matrix with about 2 column volumes of a sodium caprylate wash buffer comprising about 300 mM sodium caprylate.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; and washing the chromatography matrix with about 2 column volumes of an NaCl buffer comprising about 50 mM Tris and about 1 M NaCl, followed by about 2 column volumes of a sodium caprylate wash buffer comprising about 50 mM Tris and about 300 mM sodium caprylate.

In an embodiment, the method further comprises equilibrating the chromatography matrix with a Tris buffer at a pH of 7-9 after the mixture is applied to the chromatography column. In an embodiment, the Tris buffer comprises 10-100 mM Tris. In an embodiment, the Tris buffer comprises about 50 mM Tris. In an embodiment, the Tris buffer has a pH of about 8.0.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; equilibrating the chromatography matrix with a Tris buffer comprising about 50 mM Tris with a pH of about 8.0, and washing the chromatography matrix with about 2 column volumes of an NaCl buffer comprising about 50 mM Tris and about 1 M NaCl, followed by about 2 column volumes of a sodium caprylate wash buffer comprising about 50 mM Tris and about 300 mM sodium caprylate.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; equilibrating the chromatography matrix with a Tris buffer comprising about 50 mM Tris with a pH of about 8.0, washing the chromatography matrix with: about 2 column volumes of an NaCl buffer comprising about 50 mM Tris and about 1 M NaCl, followed by about 2 column volumes of a sodium caprylate wash buffer comprising about 50 mM Tris and about 300 mM sodium caprylate, and contacting the chromatography matrix with an elution buffer comprising about 10 mM sodium citrate with a pH of about 3.0 to produce an eluate containing dulaglutide.

In an aspect, provided herein is a method of purifying an Fc-containing protein from a mixture of Fc-containing protein and one or more HCP, the method comprising the steps of: applying the mixture to a chromatography column comprising a Protein A chromatography matrix, under conditions whereby the Fc-containing protein binds to the chromatography matrix; equilibrating the chromatography matrix with a Tris buffer comprising about 50 mM Tris with a pH of about 8.0, washing the chromatography matrix with: about 2 column volumes of an NaCl buffer comprising about 50 mM Tris and about 1 M NaCl, followed by about 2 column volumes of a sodium caprylate wash buffer comprising about 50 mM Tris and about 300 mM sodium caprylate, and contacting the chromatography matrix with an elution buffer comprising about 10 mM sodium citrate with a pH of about 3.0 to produce an eluate containing Fc-containing protein.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; equilibrating the chromatography matrix with a Tris buffer comprising about 50 mM Tris with a pH of about 8.0, washing the chromatography matrix with: about 2 column volumes of an NaCl buffer comprising about 50 mM Tris and about 1 M NaCl with a pH of about 8.0, followed by about 2 column volumes of a sodium caprylate wash buffer comprising about 50 mM Tris and about 300 mM sodium caprylate with a pH of about 8.0, and contacting the chromatography matrix with an elution buffer comprising about 10 mM sodium citrate with a pH of about 3.0 to produce an eluate containing dulaglutide.

### Protein A Chromatography

The methods provided herein generally comprise applying a mixture of an Fc-containing protein and one or more contaminant to a chromatography column comprising a protein A chromatography matrix under conditions such that the Fc-containing protein binds to the protein A chromatography matrix, and washing the protein A chromatography matrix with a sodium caprylate wash buffer (e.g., a sodium caprylate wash buffer comprising 150-500 mM sodium caprylate). The methods disclosed herein purify the Fc-containing protein (e.g., dulaglutide) from the one or more contaminant (e.g., an HCP).

The contaminant can be any material present at any stage of a method disclosed herein that is not the desired Fc-containing protein. Contaminants include, without limitation, viral and cellular proteins or nucleic acids, or byproducts thereof, that arise in the production process of the Fc-containing protein. Contaminants also include any undesired byproducts of the Fc-containing protein (e.g., fragments of the Fc-containing protein).

In an aspect, a method of the present disclosure provides improved purification of desired Fc-containing proteins from one or more HCP, resulting in an eluate that comprises a lower amount of HCPs compared to the mixture applied to the protein A chromatography matrix.

In an aspect, a method of the present disclosure provides improved purification of dulaglutide from one or more HCP, resulting in an eluate that comprises a lower amount of HCPs compared to the mixture applied to the protein A chromatography matrix.

In an aspect, the methods disclosed herein employ a protein A chromatography matrix.

In an embodiment, the chromatography column has a diameter of 10-150 cm. In an embodiment, the chromatography column has a diameter of about 1 cm, about 2 cm, about 3 cm, about 4 cm, about 5 cm, about 6 cm, about 7 cm, about 8 cm, about 9 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, about 100 cm, about 110 cm, about 120 cm, about 130 cm, about 140 cm, or about 150 cm.

In an embodiment, the chromatography column has a bed height of 10-40 cm. In an embodiment, the chromatography column has a bed height of about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 30.5 cm, about 31 cm, about 31.5 cm, about 32 cm, about 32.5 cm, about 33 cm, about 33.5 cm, about 34 cm, about 34.5 cm, about 35 cm, about 35.5 cm, about 36 cm, about 36.5 cm, about 37 cm, about 37.5 cm, about 38 cm, about 38.5 cm, about 39 cm, about 39.5 cm, or about 40 cm.

In an embodiment, the chromatography column is loaded at a temperature of about 10-40° C. In an embodiment, the chromatography column is loaded at a temperature of about 15-35° C. In an embodiment, the chromatography column is loaded at a temperature of about 15-30° C.

In an embodiment, the Protein A chromatography matrix has an average particle size of 80-90 μm. In an embodiment, the Protein A chromatography matrix has an average particle size of about 80 μm, about 81 μm, about 82 μm, about 83 μm, about 84 μm, about 85 μm, about 86 μm, about 87 μm, about 88 μm, about 89 μm, or about 90 μm. In an embodiment, the Protein A chromatography matrix has an average particle size of about 85 μm.

In an embodiment, the Protein A chromatography matrix has an average particle size of 50-70 μm. In an embodiment, the Protein A chromatography matrix has an average particle size of about 60 μm.

In an embodiment, the protein A chromatography matrix is stable in alkali. In an embodiment, the protein A chromatography matrix comprises an engineered variant of protein A that is more stable in alkali than wild-type protein A. In an embodiment, the protein A chromatography matrix comprises an engineered variant of protein A that is modified to substitute particular amino acids that are sensitive to alkali with amino acids that are more stable in alkali.

Various protein A chromatography matrices are available that are suitable for use in the methods disclosed herein. The protein A chromatography matrices can have various backbone compositions including, for example, glass or silica-based matrices, agarose-based matrices, and organic polymer-based matrices. In an embodiment, the protein A chromatography matrix comprises an engineered variant of protein A. In an embodiment, the protein A amino acid sequence comprises a C-terminal cysteine for cross-linking to a matrix. In an embodiment, the protein A chromatography matrix is an agarose matrix. In an embodiment, the protein A chromatography matrix comprises protein A tetramers cross-linked to the agarose matrix via the C-terminal cysteine on protein A. In an embodiment, the protein A chromatography matrix comprises protein A tetramers cross-linked to the agarose matrix via an epoxide linkage.

In an embodiment, the protein A chromatography matrix is a MabSelect™ protein A chromatography matrix from Cytiva (Marlborough, MA). In an embodiment, the MabSelect™ protein A chromatography matrix is MabSelect SuRe™, MabSelect SuRe™ LX, Mab Select SuRe™ pcc, or MabSelect PrismA™.

In an embodiment, the present disclosure provides a composition comprising a protein A chromatography matrix and a mixture comprising an Fc-containing protein and one or more contaminant. In an embodiment, the present disclosure provides a composition comprising a protein A chromatography matrix and a mixture comprising dulaglutide and one or more HCP. Various components of the mixture are further described herein.

### Mixtures

Methods of the present disclosure comprise contacting a mixture of the Fc-containing protein and at least one contaminant with a protein A chromatography matrix under conditions such that the Fc-containing protein binds to the protein A chromatography matrix.

In an aspect, the methods of the present disclosure comprise contacting a mixture of dulaglutide and at least one contaminant with a protein A chromatography matrix under conditions such that the Fc-containing protein binds to the protein A chromatography matrix.

In an embodiment, the contaminant is one or more HCP.

In an embodiment, the contaminant is leached Protein A, a host cell nucleic acid, a fragment of the Fc-containing protein, aggregate of the Fc-containing protein, or derivative of the of the Fc-containing protein, an endotoxin, a viral contaminant, or a cell culture media component.

Methods of the present disclosure comprise contacting a mixture of the Fc-containing protein and one or more HCP with a protein A chromatography matrix under conditions such that the Fc-containing protein binds to the protein A chromatography matrix.

In an aspect, the methods of the present disclosure comprise contacting a mixture of dulaglutide and one or more HCP with a protein A chromatography matrix under conditions such that the Fc-containing protein binds to the protein A chromatography matrix.

In an embodiment, the one or more HCP is a protease selected from the group consisting of serine protease, aspartic protease, cysteine protease, metalloprotease, and aminopeptidase, or a combination thereof.

In an embodiment, the HCP is selected from the group consisting of protein S100-A6, lysosomal acid lipase/cholesteryl ester hydrolase, C-C motif chemokine 2, phospholipid transfer protein isoform X2, sulfhydryl oxidase 1 isoform X1, farnesyl pyrophosphate synthase isoform X1, retinoid-inducible serine carboxypeptidase isoform X1, T-complex protein 1 subunit delta, 60S ribosomal protein L18 isoform X1, cytoplasmic dynein 1 heavy chain 1 isoform X1, clathrin heavy chain 1 isoform X1, metalloproteinase inhibitor 1, pigment epithelium-derived factor isoform X1, acid ceramidase isoform X1, coatomer subunit beta isoform X1, 60S ribosomal protein L10a isoform X1, cullin-associated NEDD8-dissociated protein 1, alpha-L-iduronidase isoform X1, torsin-1B-like isoform X1. In an embodiment, the contaminant HCP is cathepsin D.

In an embodiment, the host cell proteins are from a mammalian host cell. In an embodiment, the host cell is a Chinese Hamster Ovary (CHO) cell, a baby hamster kidney (BHK) cell, a murine hybridoma cell, a HEK cell, or a murine myeloma cell.

### Wash Buffers and Methods

After a mixture is applied to a chromatography column comprising a Protein A chromatography matrix, the Protein A chromatography matrix is washed with one or more wash buffers to remove one or more contaminant. In an aspect, after a mixture comprising dulaglutide and one or more HCP is applied to a chromatography column comprising a Protein A chromatography matrix, the Protein A chromatography matrix is washed with one or more wash buffers to remove one or more HCP. In an embodiment, the Protein A chromatography matrix is washed with a sodium caprylate wash buffer (e.g., a sodium caprylate wash buffer comprising 150-500 mM sodium caprylate).

Sodium caprylate (also known as sodium octanoate) is an eight-carbon saturated fatty acid with a critical micelle concentration of approximately 360 mM (chemical formula: C₈H₁₅N₈O₂).

In an aspect, provided herein is a method of purifying an Fc-containing protein from a mixture of the Fc-containing protein and one or more contaminant, the method comprising the steps of: applying the mixture to a chromatography column comprising a Protein A chromatography matrix, under conditions whereby the Fc-containing protein binds to the chromatography matrix; and washing the chromatography matrix with a sodium caprylate wash buffer comprising 150-500 mM sodium caprylate.

In an aspect, provided herein is a method of purifying dulaglutide from a mixture of dulaglutide and one or more contaminant, the method comprising the steps of: applying the mixture to a chromatography column comprising a Protein A chromatography matrix, under conditions whereby dulaglutide binds to the chromatography matrix; and washing the chromatography matrix with a sodium caprylate wash buffer comprising 150-500 mM sodium caprylate.

In an embodiment, the sodium caprylate wash buffer comprises about 150-500 mM sodium caprylate. In an embodiment, the sodium caprylate wash buffer comprises about 200-500 mM sodium caprylate. In an embodiment, the sodium caprylate wash buffer comprises about 150-350 mM sodium caprylate. In an embodiment, the sodium caprylate wash buffer comprises about 200-350 mM sodium caprylate. In an embodiment, the sodium caprylate wash buffer comprises about 250-350 mM sodium caprylate. In an embodiment, the sodium caprylate wash buffer comprises about 275-325 mM sodium caprylate.

In an embodiment, the sodium caprylate wash buffer comprises about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM sodium caprylate. In an embodiment, the sodium caprylate wash buffer comprises about 300 mM sodium caprylate.

In an embodiment, the sodium caprylate wash buffer comprises about 10-100 mM Tris. In an embodiment, the sodium caprylate wash buffer comprises about 10 mM, about 20 mM, about 30 mM, about 40 mM, about 50 mM, about 60 mM, about 70 mM, about 80 mM, about 90 mM, or about 100 mM Tris. In an embodiment, the sodium caprylate wash buffer comprises about 50 mM Tris.

In an embodiment, the sodium caprylate wash buffer comprises about 50 mM Tris and about 150-500 mM sodium caprylate. In an embodiment, the sodium caprylate wash buffer comprises about 50 mM Tris and about 200-500 mM sodium caprylate. In an embodiment, the sodium caprylate wash buffer comprises about 50 mM Tris and about 150-350 mM sodium caprylate. In an embodiment, the sodium caprylate wash buffer comprises about 50 mM Tris and about 200-350 mM sodium caprylate. In an embodiment, the sodium caprylate wash buffer comprises about 50 mM Tris and about 250-350 mM sodium caprylate. In an embodiment, the sodium caprylate wash buffer comprises about 50 mM Tris and about 275-325 mM sodium caprylate.

In an embodiment, the sodium caprylate wash buffer comprises about 50 mM Tris and about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM sodium caprylate. In an embodiment, the sodium caprylate wash buffer comprises about 50 mM Tris and about 300 mM sodium caprylate.

In an embodiment, the pH of the sodium caprylate wash buffer is about 7 to about 9. In an embodiment, the pH of the sodium caprylate wash buffer is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In an embodiment, the pH of the sodium caprylate wash buffer is about 8.

In an embodiment, the sodium caprylate wash buffer comprises about 50 mM Tris and about 150 mM sodium caprylate, and has a pH of about 8. In an embodiment, the sodium caprylate wash buffer comprises about 50 mM Tris and about 200 mM sodium caprylate, and has a pH of about 8. In an embodiment, the sodium caprylate wash buffer comprises about 50 mM Tris and about 250 mM sodium caprylate, and has a pH of about 8. In an embodiment, the sodium caprylate wash buffer comprises about 50 mM Tris and about 300 mM sodium caprylate, and has a pH of about 8. In an embodiment, the sodium caprylate wash buffer comprises about 50 mM Tris and about 350 mM sodium caprylate, and has a pH of about 8.

In an embodiment, the sodium caprylate wash buffer comprises about 0.1-2 M NaCl. In an embodiment, the sodium caprylate wash buffer comprises about 0.1 M, about 0.2 M, about 0.3 M, about 0.4 M, about 0.5 M, about 0.6 M, about 0.7 M, about 0.8 M, about 0.9 M, about 1.0 M, about 1.1 M, about 1.2 M, about 1.3 M, about 1.4 M, about 1.5 M, about 1.6 M, about 1.7 M, about 1.8 M, about 1.9 M, or about 2.0 M NaCl. In an embodiment, the sodium caprylate wash buffer comprises about 1M NaCl.

In an embodiment, the Protein A chromatography matrix is washed with an NaCl wash buffer comprising 0.5-1.5 M NaCl and a sodium caprylate wash buffer comprising 150-500 mM sodium caprylate. In an embodiment, the chromatography column is washed with the NaCl wash buffer before or after the chromatography matrix is washed with the sodium caprylate wash buffer.

In an embodiment, the NaCl wash buffer comprises about 0.1-2 M NaCl. In an embodiment, the NaCl wash buffer comprises about 0.1 M, about 0.2 M, about 0.3 M, about 0.4 M, about 0.5 M, about 0.6 M, about 0.7 M, about 0.8 M, about 0.9 M, about 1.0 M, about 1.1 M, about 1.2 M, about 1.3 M, about 1.4 M, about 1.5 M, about 1.6 M, about 1.7 M, about 1.8 M, about 1.9 M, or about 2.0 M NaCl. In an embodiment, the NaCl wash buffer comprises about 1M NaCl.

In an embodiment, the NaCl wash buffer comprises about 10-100 mM Tris. In an embodiment, the NaCl wash buffer comprises about 10 mM, about 20 mM, about 30 mM, about 40 mM, about 50 mM, about 60 mM, about 70 mM, about 80 mM, about 90 mM, or about 100 mM Tris. In an embodiment, the NaCl wash buffer comprises about 50 mM Tris.

In an embodiment, the NaCl wash buffer comprises about 50 mM Tris and about 0.1-2 M NaCl. In an embodiment, the NaCl wash buffer comprises about 50 mM Tris and about 0.1 M, about 0.2 M, about 0.3 M, about 0.4 M, about 0.5 M, about 0.6 M, about 0.7 M, about 0.8 M, about 0.9 M, about 1.0 M, about 1.1 M, about 1.2 M, about 1.3 M, about 1.4 M, about 1.5 M, about 1.6 M, about 1.7 M, about 1.8 M, about 1.9 M, or about 2.0 M NaCl. In an embodiment, the NaCl wash buffer comprises about 50 mM Tris and about 1M NaCl.

In an embodiment, the pH of the NaCl wash buffer is about 7 to about 9. In an embodiment, the pH of the NaCl wash buffer is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In an embodiment, the pH of the NaCl wash buffer is about 8.

In an embodiment, the NaCl wash buffer comprises about 50 mM Tris and about 0.8 M NaCl, and has a pH of about 8. In an embodiment, the sodium caprylate wash buffer comprises about 50 mM Tris and about 0.9 M NaCl, and has a pH of about 8. In an embodiment, the sodium caprylate wash buffer comprises about 50 mM Tris and about 1.0 M NaCl, and has a pH of about 8. In an embodiment, the sodium caprylate wash buffer comprises about 50 mM Tris and about 1.1 M NaCl, and has a pH of about 8.

In an embodiment, the NaCl wash buffer comprises about 50 mM Tris and about 0.8 M NaCl, and has a pH of about 8; and the sodium caprylate wash buffer comprises about 50 mM Tris and about 150 mM sodium caprylate, and has a pH of about 8. In an embodiment, the NaCl wash buffer comprises about 50 mM Tris and about 0.9 M NaCl, and has a pH of about 8; and the sodium caprylate wash buffer comprises about 50 mM Tris and about 150 mM sodium caprylate, and has a pH of about 8. In an embodiment, the NaCl wash buffer comprises about 50 mM Tris and about 1.0 M NaCl, and has a pH of about 8; and the sodium caprylate wash buffer comprises about 50 mM Tris and about 150 mM sodium caprylate, and has a pH of about 8. In an embodiment, the NaCl wash buffer comprises about 50 mM Tris and about 1.1 M NaCl, and has a pH of about 8; and the sodium caprylate wash buffer comprises about 50 mM Tris and about 150 mM sodium caprylate, and has a pH of about 8.

In an embodiment, the NaCl wash buffer comprises about 50 mM Tris and about 0.8 M NaCl, and has a pH of about 8; and the sodium caprylate wash buffer comprises about 50 mM Tris and about 200 mM sodium caprylate, and has a pH of about 8. In an embodiment, the NaCl wash buffer comprises about 50 mM Tris and about 0.9 M NaCl, and has a pH of about 8; and the sodium caprylate wash buffer comprises about 50 mM Tris and about 200 mM sodium caprylate, and has a pH of about 8. In an embodiment, the NaCl wash buffer comprises about 50 mM Tris and about 1.0 M NaCl, and has a pH of about 8; and the sodium caprylate wash buffer comprises about 50 mM Tris and about 200 mM sodium caprylate, and has a pH of about 8. In an embodiment, the NaCl wash buffer comprises about 50 mM Tris and about 1.1 M NaCl, and has a pH of about 8; and the sodium caprylate wash buffer comprises about 50 mM Tris and about 200 mM sodium caprylate, and has a pH of about 8.

In an embodiment, the NaCl wash buffer comprises about 50 mM Tris and about 0.8 M NaCl, and has a pH of about 8; and the sodium caprylate wash buffer comprises about 50 mM Tris and about 250 mM sodium caprylate, and has a pH of about 8. In an embodiment, the NaCl wash buffer comprises about 50 mM Tris and about 0.9 M NaCl, and has a pH of about 8; and the sodium caprylate wash buffer comprises about 50 mM Tris and about 250 mM sodium caprylate, and has a pH of about 8. In an embodiment, the NaCl wash buffer comprises about 50 mM Tris and about 1.0 M NaCl, and has a pH of about 8; and the sodium caprylate wash buffer comprises about 50 mM Tris and about 250 mM sodium caprylate, and has a pH of about 8. In an embodiment, the NaCl wash buffer comprises about 50 mM Tris and about 1.1 M NaCl, and has a pH of about 8; and the sodium caprylate wash buffer comprises about 50 mM Tris and about 250 mM sodium caprylate, and has a pH of about 8.

In an embodiment, the NaCl wash buffer comprises about 50 mM Tris and about 0.8 M NaCl, and has a pH of about 8; and the sodium caprylate wash buffer comprises about 50 mM Tris and about 300 mM sodium caprylate, and has a pH of about 8. In an embodiment, the NaCl wash buffer comprises about 50 mM Tris and about 0.9 M NaCl, and has a pH of about 8; and the sodium caprylate wash buffer comprises about 50 mM Tris and about 300 mM sodium caprylate, and has a pH of about 8. In an embodiment, the NaCl wash buffer comprises about 50 mM Tris and about 1.0 M NaCl, and has a pH of about 8; and the sodium caprylate wash buffer comprises about 50 mM Tris and about 300 mM sodium caprylate, and has a pH of about 8. In an embodiment, the NaCl wash buffer comprises about 50 mM Tris and about 1.1 M NaCl, and has a pH of about 8; and the sodium caprylate wash buffer comprises about 50 mM Tris and about 300 mM sodium caprylate, and has a pH of about 8.

In an embodiment, the NaCl wash buffer comprises about 50 mM Tris and about 0.8 M NaCl, and has a pH of about 8; and the sodium caprylate wash buffer comprises about 50 mM Tris and about 350 mM sodium caprylate, and has a pH of about 8. In an embodiment, the NaCl wash buffer comprises about 50 mM Tris and about 0.9 M NaCl, and has a pH of about 8; and the sodium caprylate wash buffer comprises about 50 mM Tris and about 350 mM sodium caprylate, and has a pH of about 8. In an embodiment, the NaCl wash buffer comprises about 50 mM Tris and about 1.0 M NaCl, and has a pH of about 8; and the sodium caprylate wash buffer comprises about 50 mM Tris and about 350 mM sodium caprylate, and has a pH of about 8. In an embodiment, the NaCl wash buffer comprises about 50 mM Tris and about 1.1 M NaCl, and has a pH of about 8; and the sodium caprylate wash buffer comprises about 50 mM Tris and about 350 mM sodium caprylate, and has a pH of about 8.

In an embodiment, the chromatography matrix is washed with about 1-10 column volumes of the NaCl wash buffer. In an embodiment, the chromatography matrix is washed with about 1, about 2, about 3, about 4, about 5, about 6, about 7, about 8, about 9, or about 10 column volumes of the NaCl wash buffer.

In an embodiment, the chromatography matrix is equilibrated with a Tris buffer at a pH of about 7-9 after the mixture is applied to the chromatography column and before the chromatography matrix is washed.

In an embodiment, the Tris buffer comprises about 10-100 mM Tris. In an embodiment, the Tris buffer comprises about 10 mM, about 20 mM, about 30 mM, about 40 mM, about 50 mM, about 60 mM, about 70 mM, about 80 mM, about 90 mM, or about 100 mM Tris. In an embodiment, the Tris buffer comprises about 50 mM Tris.

In an embodiment, the Tris buffer comprises about 10-100 mM Tris and has a pH of about 8. In an embodiment, the Tris buffer comprises about 10 mM, about 20 mM, about 30 mM, about 40 mM, about 50 mM, about 60 mM, about 70 mM, about 80 mM, about 90 mM, or about 100 mM Tris and has a pH of about 8. In an embodiment, the Tris buffer comprises about 50 mM Tris and has a pH of about 8.

In an embodiment, the chromatography matrix is washed with about 1-10 column volumes of the sodium caprylate wash buffer. In an embodiment, the chromatography matrix is washed with about 1, about 2, about 3, about 4, about 5, about 6, about 7, about 8, about 9, or about 10 column volumes of the sodium caprylate wash buffer.

In an embodiment, the chromatography matrix is washed with about 1-10 column volumes of the NaCl wash buffer followed by about 1 column volume of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 1 column volume of the sodium caprylate wash buffer.

In an embodiment, the chromatography matrix is washed with about 1-10 column volumes of the NaCl wash buffer followed by about 2 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 2 column volumes of the sodium caprylate wash buffer.

In an embodiment, the chromatography matrix is washed with about 1-10 column volumes of the NaCl wash buffer followed by about 3 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 3 column volumes of the sodium caprylate wash buffer.

In an embodiment, the chromatography matrix is washed with about 1-10 column volumes of the NaCl wash buffer followed by about 4 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 4 column volumes of the sodium caprylate wash buffer.

In an embodiment, the chromatography matrix is washed with about 1-10 column volumes of the NaCl wash buffer followed by about 5 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 5 column volumes of the sodium caprylate wash buffer.

In an embodiment, the chromatography matrix is washed with about 1-10 column volumes of the NaCl wash buffer followed by about 6 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 6 column volumes of the sodium caprylate wash buffer.

In an embodiment, the chromatography matrix is washed with about 1-10 column volumes of the NaCl wash buffer followed by about 7 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 7 column volumes of the sodium caprylate wash buffer.

In an embodiment, the chromatography matrix is washed with about 1-10 column volumes of the NaCl wash buffer followed by about 8 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 8 column volumes of the sodium caprylate wash buffer.

In an embodiment, the chromatography matrix is washed with about 1-10 column volumes of the NaCl wash buffer followed by about 9 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 9 column volumes of the sodium caprylate wash buffer.

In an embodiment, the chromatography matrix is washed with about 1-10 column volumes of the NaCl wash buffer followed by about 10 column volumes of the sodium caprylate wash buffer. In an embodiment, the chromatography matrix is washed with about 1, about 2, about 3, about 4, about 5, about 6, about 7, about 8, about 9, or about 10 column volumes of the NaCl wash buffer followed by about 10 column volumes of the sodium caprylate wash buffer.

In an embodiment, the chromatography matrix is washed with the NaCl wash buffer at a flow rate of about 100-400 cm/hr. In an embodiment, the chromatography matrix is washed with the NaCl wash buffer at a flow rate of about 100 cm/hr, about 150 cm/hr, about 200 cm/hr, about 250 cm/hr, about 300 cm/hr, about 350 cm/hr, or about 400 cm/hr. In an embodiment, the chromatography matrix is washed with the NaCl wash buffer at a flow rate of 300±30 cm/hr.

In an embodiment, the chromatography matrix is washed with the sodium caprylate wash buffer at a flow rate of about 100-400 cm/hr. In an embodiment, the chromatography matrix is washed with the sodium caprylate wash buffer at a flow rate of about 100 cm/hr, about 150 cm/hr, about 200 cm/hr, about 250 cm/hr, about 300 cm/hr, about 350 cm/hr, or about 400 cm/hr. In an embodiment, the chromatography matrix is washed with the sodium caprylate wash buffer at a flow rate of 300±30 cm/hr.

In an embodiment, the chromatography matrix is washed with the NaCl wash buffer at a flow rate of 300±30 cm/hr and the sodium caprylate wash buffer at a flow rate of about 100-400 cm/hr. In an embodiment, the chromatography matrix is washed with the NaCl wash buffer at a flow rate of 300±30 cm/hr and the sodium caprylate wash buffer at a flow rate of about 100 cm/hr, about 150 cm/hr, about 200 cm/hr, about 250 cm/hr, about 300 cm/hr, about 350 cm/hr, or about 400 cm/hr. In an embodiment, the chromatography matrix is washed with the NaCl wash buffer at a flow rate of 300±30 cm/hr and the sodium caprylate wash buffer at a flow rate of 300±30 cm/hr.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; and washing the chromatography matrix with: about 2 column volumes of an NaCl buffer comprising about 1 M NaCl, followed by about 2 column volumes of a sodium caprylate wash buffer comprising about 300 mM sodium caprylate, thereby purifying the dulaglutide from one or more HCP in the mixture.

In an aspect, provided herein is a method of purifying dulaglutide from a mixture of dulaglutide and one or more HCP, the method comprising the steps of: applying the mixture to a chromatography column comprising a Protein A chromatography matrix, under conditions whereby dulaglutide binds to the chromatography matrix; and washing the chromatography matrix with: about 2 column volumes of an NaCl buffer comprising about 50 mM Tris and about 1 M NaCl at a flow rate of 300±30 cm/hr, followed by about 2 column volumes of a sodium caprylate wash buffer comprising about 50 mM Tris and about 300 mM sodium caprylate at a flow rate of 300±30 cm/hr, thereby purifying the dulaglutide from one or more HCP in the mixture.

In any one of the methods disclosed herein, the method further comprises washing the chromatography matrix with a Tris buffer comprising 10-100 mM Tris buffer after washing with the sodium caprylate buffer. In an embodiment, the Tris buffer comprises about 10-100 mM Tris and has a pH of about 8. In an embodiment, the Tris buffer comprises about 10 mM, about 20 mM, about 30 mM, about 40 mM, about 50 mM, about 60 mM, about 70 mM, about 80 mM, about 90 mM, or about 100 mM Tris and has a pH of about 8. In an embodiment, the Tris buffer comprises about 50 mM Tris and has a pH of about 8.

Elution from Protein A

Methods of the present disclosure further comprise eluting the Fc-containing protein from the protein A chromatography matrix. An Fc-containing protein bound to the protein A chromatography matrix can be eluted using an acid or a combination of acids (e.g., a weak acid and a strong acid).

Elution of the Fc-containing protein (e.g., dulaglutide) bound to the protein A chromatography matrix is achieved by contacting the protein A chromatography matrix with an elution buffer to produce an eluate.

In an embodiment, the protein A chromatography matrix is rinsed with a Tris buffer comprising about 50 mM Tris with a pH of about 8.0 prior to the elution step.

In an embodiment, the elution buffer comprises sodium citrate. In an embodiment, the elution buffer comprises about 5-25 mM sodium citrate. In an embodiment, the elution buffer comprises about 10 mM sodium citrate. In an embodiment, the elution buffer comprises about 5, mM, about 6 mM, about 7 mM, about 8 mM, about 9 mM, about 10 mM, about 11 mM, about 12, mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM sodium citrate.

In an embodiment, the elution buffer has a pH of about 2.5-4. In an embodiment, the elution buffer has a pH of about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0.

In an embodiment, the elution buffer comprises about 10 mM sodium citrate and has a pH of 2.5-4. In an embodiment, the elution buffer comprises about 10 mM sodium citrate and has a pH of about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0.

The skilled artisan will appreciate that the elution buffer may comprise additional components that aid in the elution of the Fc-containing protein from the protein A chromatography matrix. Such components may include additional buffering agents and additives that aid in, for example, dissociation and solubilization.

In an embodiment, the eluate is monitored by absorbance using a spectrophotometer.

In an embodiment, the eluate comprises at least about 70%, at least about 75%, at least about 80%, at least about 85%, at least about 90%, at least about 95%, at least about 96%, at least about 97%, at least about 98%, at least about 99% of the Fc-containing protein that was in the mixture. The percent recovery can be determined, for example, by calculating the percentage of Fc-containing protein in the eluate relative to the amount that was in the mixture applied to the chromatography column.

In an embodiment, the eluate comprises at least about 70%, at least about 75%, at least about 80%, at least about 85%, at least about 90%, at least about 95%, at least about 96%, at least about 97%, at least about 98%, at least about 99% of the dulaglutide that was in the mixture. The percent recovery can be determined, for example, by calculating the percentage of dulaglutide in the eluate relative to the amount that was in the mixture applied to the chromatography column.

In an embodiment, the eluate is applied to another chromatography matrix to further purify dulaglutide from any residual contaminants (e.g., HCPs). In an embodiment, the eluate is applied to an anion exchange (AEX) chromatography matrix, a size exclusion, hydrophobic interaction, ion exchange, or hydroxyapatite chromatography.

### Host Cell Protein Detection

The methods provided herein result in a significant reduction in host cell proteins (HCPs) after protein A purification of an Fc-containing protein (e.g., dulaglutide) compared to other methods known in the art.

In an embodiment, the one or more HCP comprise is a protease selected from the group consisting of serine protease, aspartic protease, cysteine protease, metalloprotease, and aminopeptidase, or a combination thereof.

In an embodiment, the HCP is selected from the group consisting of protein S100-A6, lysosomal acid lipase/cholesteryl ester hydrolase, C-C motif chemokine 2, phospholipid transfer protein isoform X2, sulfhydryl oxidase 1 isoform X1, farnesyl pyrophosphate synthase isoform X1, retinoid-inducible serine carboxypeptidase isoform X1, T-complex protein 1 subunit delta, 60S ribosomal protein L18 isoform X1, cytoplasmic dynein 1 heavy chain 1 isoform X1, clathrin heavy chain 1 isoform X1, metalloproteinase inhibitor 1, pigment epithelium-derived factor isoform X1, acid ceramidase isoform X1, coatomer subunit beta isoform X1, 60S ribosomal protein L10a isoform X1, cullin-associated NEDD8-dissociated protein 1, alpha-L-iduronidase isoform X1, torsin-1B-like isoform X1, and cathepsin D. In an embodiment, the HCP is cathepsin D.

Methods for determining the host cell protein (HCP) concentration are known in the art. In an embodiment, an immunoassay is used to detect the amount of HCPs in an eluate. In an embodiment, the immunoassay is an enzyme-linked immunosorbent assay (ELISA). In an HCP ELISA, the primary antibody is specific to the HCPs produced from a particular host cell, e.g., CHO cells, used to generate the Fc-containing protein. In an embodiment, the ELISA is a Gyrolab® CHO-HCP Kit 1 (Cygnus Technologies, Warren, NJ) ELISA assay.

In an embodiment, the amount of HCPs in an eluate is measured by mass spectrometry. In an embodiment, the mass spectrometry analysis is LC-MS. In an LC-MS assay samples are analyzed by peptide mapping/LC-MS/MS HCP profiling via, e.g., an Ultra Performance Liquid Chromatography (UPLC) coupled to a Thermo Scientific mass spectrometer. In this analysis, the samples are subjected to digestion by trypsin, reduced/precipitated with dithiothreitol (DTT), followed by transfer and acidification of the supernatant in a HPLC vial for LC-MS/MS analysis. LC-MS/MS data can be analyzed by Proteome Discoverer against a CHO-K1 protein database. The HCP content is reported as total parts per million (ppm) of HCP per sample for total HCP content (e.g., ng of HCP per mg of product).

Methods for determining the level of cathepsin D activity in an eluate are known in the art. In general, any assay that can reliably detect cathepsin D activity may be used.

In an embodiment, the eluate comprises a reduced amount of one or more HCP compared to the mixture. In an embodiment, the eluate comprises a reduced amount of cathepsin D compared to the mixture. In an embodiment, the eluate comprises reduced cathepsin D activity compared to the mixture.

In an embodiment, the eluate comprises less than about 100 ng/mg HCPs. In an embodiment, the eluate comprises less than about 90 ng/mg, less than about 80 ng/mg, less than about 70 ng/mg, less than about 60 ng/mg, less than about 50 ng/mg, or less than about 40 ng/mg.

In an embodiment, the eluate comprises less than about 100 ppm, less than about 90 ppm, less than about 80 ppm, less than about 70 ppm, less than about 60 ppm, less than about 50 ppm, less than about 40 ppm, less than about 30 ppm, or to less than about 20 ppm HCPs.

In an embodiment, the eluate comprises less than about 90 ng/mg, less than about 80 ng/mg, less than about 70 ng/mg, less than about 60 ng/mg, less than about 50 ng/mg, less than about 40 ng/mg, less than about 30 ng/mg, less than about 20 ng/mg, less than about 10 ng/mg, less than about 9 ng/mg, less than about 8 ng/mg, less than about 7 ng/mg, less than about 6 ng/mg, less than about 5 ng/mg, less than about 4 ng/mg, less than about 3 ng/mg, less than about 2 ng/mg, or less than about 1 ng/mg cathepsin D.

In an embodiment, the eluate comprises less than about 100 ppm, less than about 90 ppm, less than about 80 ppm, less than about 70 ppm, less than about 60 ppm, less than about 50 ppm, less than about 40 ppm, less than about 30 ppm, or to less than about 20 ppm cathepsin D.

In an embodiment, the amount of HCPs is measured by mass spectrometry or ELISA. In an embodiment, the amount of cathepsin D is measured by mass spectrometry or ELISA. In an embodiment, the mass spectrometry is LC-MS.

In an embodiment, the eluate comprises less than 300 μU/mL cathepsin D activity. In an embodiment, the eluate comprises less than 350 μU/mL, less than 325 μU/mL, less than 300 μU/mL, less than 275 μU/mL, less than 250 μU/mL, less than 225 μU/mL, less than 200 μU/mL, less than 175 μU/mL, less than 150 μU/mL, less than 125 μU/mL, less than 100 μU/mL cathepsin D activity, or less than 50 μU/mL cathepsin D activity.

### III. Fc-Containing Proteins

The methods provided by the present disclosure are for the purification of an Fc-containing protein from a mixture of the Fc-containing protein and one or more contaminant.

In an embodiment, the Fc-containing protein was produced in mammalian host cells. In an embodiment, the Fc-containing protein was produced in Chinese Hamster Ovary (CHO) cells, baby hamster kidney (BHK) cells, murine hybridoma cells, or murine myeloma cells.

In an embodiment, the Fc-containing protein comprises one or more of the amino acid sequences set forth in Table 1 below.

In an embodiment, the Fc-containing protein comprises a glucagon-like peptide I (GLP-1) analog comprising one or more modifications compared to a wild type GLP-1 amino acid sequence (SEQ ID NO: 1).

In an embodiment, the Fc-containing protein comprises a GLP-1 analog comprising the amino acid sequence of SEQ ID NO: 2.

In an embodiment, the Fc-containing protein comprises a peptide linker. In an embodiment, the C-terminal amino acid of the GLP-1 analog portion of the Fc-containing protein is fused to the N-terminus of an Fc portion of an immunoglobulin via a peptide linker. In an embodiment, the peptide linker comprises 1 to 10 G₄S units (SEQ ID NO: 3).

In an embodiment, the Fc-containing protein comprises: a GLP-1 analog comprising the amino acid sequence of SEQ ID NO: 2; a peptide linker comprising the amino acid sequence of SEQ ID NO: 3; and an Fc portion of an immunoglobulin. In an embodiment, the N-terminal residue of the peptide linker is directly fused to the C-terminal residue of the GLP-1 analog, and the C-terminal residue of the peptide linker is directly fused to the N-terminal residue of the Fc portion.

In an embodiment, the Fc-containing protein is a homodimer comprising two identical amino acid chains each comprising the amino acid sequence of SEQ ID NO: 4.

In an embodiment, the Fc-containing protein is dulaglutide.

In an aspect, provided herein are methods for the purification of dulaglutide from a mixture of dulaglutide and one or more HCP. In an embodiment, dulaglutide is produced in CHO cells.

Dulaglutide is a human GLP-1 receptor agonist which comprises a dimer of a GLP-1 analog fused at its C-terminus via a peptide linker to the N-terminus of an analog of an Fc portion of an immunoglobulin, and is identified by CAS registry number 923950-08-7, which provides the following chemical name: 7-37-Glucagon-like peptide I [8-glycine, 22-glutamic acid, 36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO: 4.

The two monomers are attached by disulfide bonds between the cysteine residues at positions 55 and 58 of SEQ ID NO: 4 to form the dimer. Dulaglutide's structure, function, production, and use in treating types 2 diabetes mellitus (T2DM) is described in more detail in U.S. Pat. No. 7,452,966 and U.S. Patent Application Publication No. US20100196405. Dulaglutide agonizes the GLP-1 receptor resulting in stimulation of insulin synthesis and secretion and has been shown to provide improved glycemic control in T2DM patients.

When used herein, the term “dulaglutide” refers to any GLP-1 receptor agonist protein dimer of two monomers having the amino acid sequence of SEQ ID NO: 4, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term.

TABLE 1
Sequences of dulaglutide

| Description | SEQ ID NO: | Amino acid sequence |
|---|---|---|
| WT GLP 1 | 1 | HAEGTFTSDVSSYLEGQAAKEFLAWLVKGRG |
| GLP 1 | 2 | HGEGTFTSDVSSYLEEQAAKEFIAWLVKGGG |
| variant | | |
| G₄S | 3 | GGGGS |
| Dulaglutide | 4 | HGEGTFTSDVSSYLEEQAAKEFIAWLVKGGGGGGGSGGGGSGGGGSAE |
| monomer | | SKYGPPCPPCPAPEAAGGPSVFLFPPKPKDTLMISRTPEVTCVVVDVSQE |
| | | DPEVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQVSLT |
| | | CLVKGFYPSDIAVEWESNGQPENNYKTTPPVLDSDGSFFLYSRLTVDKS |
| | | RWQEGNVFSCSVMHEALHNHYTQKSLSLSLG |

In an embodiment, the Fc-containing protein is etanercept, alefacept, abatacept, rilonacept, romiplostim, belatacept, aflibercept, conbercept, efmoroctocog alpha, eftrenonacog alpha, asfotase alpha, or luspatercept.

In an aspect, provided herein is an Fc-containing protein produced by any one of the methods disclosed herein.

In an aspect, provided herein is dulaglutide produced by any one of the methods disclosed herein.

### EXAMPLES

The following examples are offered by way of illustration, and not by way of limitation.

### Example 1: Analysis of Wash Buffer Additives for Removing Host Cell Proteins (HCPs)

Protein A purification is used as a step in the manufacturing process of dulaglutide in order to reduce process related impurities such as media components, Triton X-100, HCP and DNA, and to reduce potential viral contaminants. Dulaglutide binds to the protein A chromatography matrix and the contaminants or impurities are removed by washing the matrix with a series of wash buffers. However, after protein A purification there can be residual host cell proteins (HCPs) in the eluate, which can affect product quality and patient safety by posing concerns such as aggregation, product fragmentation by catalytic activity, and/or immunogenicity. In this example, various wash conditions were tested in order to reduce the level of HCPs in the protein A eluate.

CHO cells expressing dulaglutide were harvested and clarified by methods known in the art. The dulaglutide clarified cell broth was then loaded onto a chromatography column containing MabSelect SuRe LX™ protein A matrix, using the loading conditions in the Wash Study column in Table 4 below. The column was equilibrated with 50 mM Tris pH 8.0.

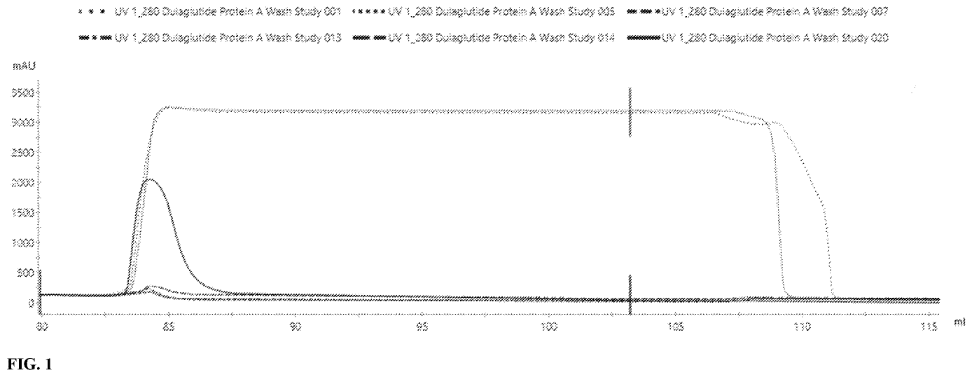

For analysis of optimal washing conditions, various buffers with buffer additives targeted towards disrupting non-covalent interactions were compared as Protein A washes. The primary objective of this study was to improve the overall clearance of host cell proteins (HCPs) in the process of dulaglutide purification. The flow throughs from these washes were monitored by a spectrophotometer to monitor for peaks, which would indicate the possible removal of unwanted substances (e.g., HCPs). The results in FIG. 1 show that washing the protein A chromatography matrix with a 50 mM Tris, pH 8.0 buffer containing 300 mM sodium caprylate as an additive resulted in a sizable UV peak at the beginning of the wash step, suggesting the possible removal of unwanted HCPs. Other additives, including 1 M sodium chloride, 1 M lysine and 1 M arginine, resulted in much smaller peaks initially. The wash buffers with sodium benzoate resulted in broad peaks throughout the entire wash, which may hide any protein breakthrough.

To analyze the impact of the wash buffer additives on HCP clearance, the protein A chromatography matrix was washed once with a wash buffer containing either 1 M sodium benzoate, 1 M NaCl, 1 M lysine, 1 M arginine, or 300 mM sodium caprylate. Dulaglutide was then eluted from protein A with an elution buffer (10 mM Sodium Citrate, pH 3.0) and the HCP level in the eluate was analyzed by ELISA and LC-MS.

For the Host cell protein (HCP) measurements by LC-MS, samples were analyzed by peptide mapping/LC-MS/MS HCP profiling via, e.g., an Ultra Performance Liquid Chromatography (UPLC) coupled to a Thermo Scientific mass spectrometer. In this analysis, the samples were subjected to digestion by trypsin, reduced/precipitated with dithiothreitol (DTT), followed by transfer and acidification of the supernatant in an HPLC vial for LC-MS/MS analysis. The LC-MS/MS data was analyzed by Proteome Discoverer against a CHO-K1 protein database with added control protein sequences. The HCP content is reported as total parts per million (ppm) of HCP per sample for total HCP content.

For the HCP ELISA, an ELISA assay was done using a Gyrolab® CHO-HCP Kit 1 (Cygnus Technologies, performed per manufacturer instructions). The HCP content is reported as ng of HCP per mg of product.

As shown in Table 2 below, 300 mM sodium caprylate as a single additive resulted in significantly reduced levels of the most immunogenic HCPs (i.e., HCPs with the highest EpiMatrix Score) compared to the other additives tested. The dulaglutide yields were similar for all wash buffers tested. Additionally, as shown in Table 3, wash buffers containing combinations of additives were tested, and wash buffers with combinations containing sodium caprylate consistently had the lowest levels of the most immunogenic HCPs.

These results demonstrate that adding sodium caprylate to the wash buffer for Protein A purification of dulaglutide can effectively reduce levels of HCPs in the protein A eluate, including the most immunogenic HCPs and thus most potentially harmful to patient safety.

TABLE 2
Host cell protein (HCP) analysis for Protein A dulaglutide wash buffer additives.

| | EpiMatrix* | 1M | 1M | 1M | 1M | 300 mM |
|---|---|---|---|---|---|---|
| HCP Species | Score | Benzoate | NaCl | Lysine | Arginine | Caprylate |
| protein S100-A6 | 52.84 | 79.6 | 75.9 | 84.2 | 107 | 66 |
| lysosomal acid lipase/ | 14.26 | 51.2 | 47.8 | 62.2 | 59.5 | 40.3 |
| cholesteryl ester hydrolase | | | | | | |
| C-C motif chemokine 2 | 18.31 | 34 | 28.3 | 30 | 38.7 | 24.4 |
| phospholipid transfer | 45.38 | 8.58 | 10.8 | 39.4 | 29.2 | 6.87 |
| protein isoform X2 | | | | | | |
| sulfhydryl oxidase 1 isoform X1 | 42.18 | 10.2 | 10.4 | 10.4 | 11.2 | 7.71 |
| farnesyl pyrophosphate | 17.6 | 8.14 | 5.85 | 5.67 | 7.31 | 0 |
| synthase isoform X1 | | | | | | |
| retinoid-inducible serine | 15.52 | 1.01 | 5.01 | 6.25 | 5.96 | 2.4 |
| carboxypeptidase isoform X1 | | | | | | |
| T-complex protein 1 | 28.05 | 3.59 | 4.69 | 6.14 | 4.87 | 2.52 |
| subunit delta | | | | | | |
| 60S ribosomal protein | 89.75 | 2.59 | 1.51 | 5.5 | 4.63 | 0 |
| L18 isoform X1 | | | | | | |
| cytoplasmic dynein 1 heavy | 16.61 | 1.55 | 2.81 | 2.98 | 3.92 | 0 |
| chain 1 isoform X1 | | | | | | |
| clathrin heavy chain 1 | 27.72 | 3.8 | 2.8 | 1.35 | 3.02 | 1.31 |
| isoform X1 | | | | | | |
| metalloproteinase | 40.09 | 3.06 | 6.4 | 6.75 | 3.01 | 6.6 |
| inhibitor 1 | | | | | | |
| pigment epithelium-derived | 49.98 | 4.92 | 1.13 | 4.82 | 2.43 | 0 |
| factor isoform X1 | | | | | | |
| acid ceramidase isoform X1 | 28.16 | 3.09 | 0 | 4.65 | 1.5 | 0.968 |
| coatomer subunit beta | 13.68 | 0 | 0 | 3.08 | 1.5 | 0 |
| isoform X1 | | | | | | |
| 60S ribosomal protein | 96.38 | 0 | 0 | 1.19 | 0.858 | 0 |
| L10a isoform X1 | | | | | | |
| cullin-associated NEDD8- | 44.48 | 0 | 0 | 0.793 | 0 | 0 |
| dissociated protein 1 | | | | | | |
| alpha-L-iduronidase | 29.17 | 0 | 1.66 | 0 | 0 | 0 |
| isoform X1 | | | | | | |
| torsin-1B-like isoform X1 | 19.23 | 0 | 2.61 | 2.73 | 0 | 1.19 |

| | | | | | |
|---|---|---|---|---|---|
| total HCP (relative ppm) | 2671 | 2629 | 3202 | 3087 | 1833 |
| Pro A Yield (%) | 97.0 | 99.9 | 104.0 | 98.8 | 100.3 |
| Residual HCP by ELISA (ng/mg) | 71 | 104 | 77 | 74 | 77 |

*EpiMatrix score expresses the T cell epitope concentration in a protein sequence.

TABLE 3
Host cell protein (HCP) analysis for Protein A dulaglutide wash buffer additive combinations.

| | | | 1M | 1M | | 1M | | 1M | 300 | 1M | |
|---|---|---|---|---|---|---|---|---|---|---|---|
| | | 1M | Benzoate, | Lysine, | 1M | Lysine, | 1M | Lysine, | mM | Benzoate, | 1M |
| | | Lysine, | 300 | 1M | Lysine, | 300 | Benzoate, | 300 | Caprylate, | 300 | Lysine, |
| Sample | | 1M | mM | Benzoate, | 1M | mM | 1M | mM | 1M | mM | 1M |
| | Epi- | Benzoate, | Caprylate, | 300 | Benzoate, | Caprylate, | Arginine, | Caprylate, | Arginine, | Caprylate, | Arginine, |
| HCP | Matrix | 1M | 1M | mM | 1M | 1M | 1M | 1M | 1M | 1M | 1M |
| Species | Score | Arginine | Arginine | Caprylate | NaCl | Arginine | NaCl | NaCl | NaCl | NaCl | NaCl |
| protein | 52.84 | 78.6 | 49.2 | 58.2 | 86.6 | 92.7 | 78.9 | 74.7 | 72.6 | 37.5 | 96 |
| S100-A6 | | | | | | | | | | | |
| lysosomal | 14.26 | 55.5 | 40.1 | 35.9 | 65.4 | 52.9 | 65.2 | 44.5 | 45.7 | 0 | 65.8 |
| acid | | | | | | | | | | | |
| lipase/ | | | | | | | | | | | |
| cholesteryl | | | | | | | | | | | |
| ester | | | | | | | | | | | |
| hydrolase | | | | | | | | | | | |
| C-C motif | 18.31 | 30.3 | 21.7 | 21.9 | 32.4 | 35.2 | 31.1 | 29.8 | 28.4 | 0 | 36.4 |
| chemokine 2 | | | | | | | | | | | |
| phospholipid | 45.38 | 11.3 | 9.35 | 8.8 | 10.9 | 10.9 | 8.66 | 8.24 | 8.38 | 0 | 36.7 |
| transfer | | | | | | | | | | | |
| protein | | | | | | | | | | | |
| isoform X2 | | | | | | | | | | | |
| sulfhydryl | 42.18 | 10.5 | 5.16 | 5.94 | 9.67 | 11.2 | 10.3 | 9.55 | 9.77 | 0 | 10.5 |
| oxidase 1 | | | | | | | | | | | |
| isoform X1 | | | | | | | | | | | |
| farnesyl | 17.6 | 0.717 | 0.435 | 1.32 | 2.87 | 0 | 2.58 | 0 | 0 | 0 | 0 |
| pyrophosphate | | | | | | | | | | | |
| synthase | | | | | | | | | | | |
| isoform X1 | | | | | | | | | | | |
| retinoid- | 15.52 | 0.931 | 2.25 | 0 | 6.25 | 3.41 | 1.03 | 3.8 | 0 | 0 | 4.18 |
| inducible | | | | | | | | | | | |
| serine | | | | | | | | | | | |
| carboxy- | | | | | | | | | | | |
| peptidase | | | | | | | | | | | |
| isoform X1 | | | | | | | | | | | |
| T-complex | 28.05 | 4.16 | 2.45 | 2.86 | 4.51 | 3.94 | 3.76 | 3.54 | 2.81 | 0 | 6.08 |
| protein 1 | | | | | | | | | | | |
| subunit | | | | | | | | | | | |
| delta | | | | | | | | | | | |
| 60S ribosomal | 89.75 | 2.63 | 0 | 0 | 2.99 | 0 | 0 | 0 | 0 | 0 | 8.28 |
| protein L18 | | | | | | | | | | | |
| isoform X1 | | | | | | | | | | | |
| cytoplasmic | 16.61 | 0 | 0 | 0 | 2.55 | 3.52 | 0.597 | 0 | 0 | 0 | 3.7 |
| dynein 1 | | | | | | | | | | | |
| heavy | | | | | | | | | | | |
| chain 1 | | | | | | | | | | | |
| isoform X1 | | | | | | | | | | | |
| clathrin | 27.72 | 0 | 0 | 0 | 0 | 0 | 0 | 0 | 0 | 0 | 0 |
| heavy | | | | | | | | | | | |
| chain 1 | | | | | | | | | | | |
| isoform X1 | | | | | | | | | | | |
| metallopro- | 40.09 | 3.18 | 2.58 | 2.63 | 9 | 3.28 | 2.57 | 3.16 | 2.48 | 0 | 9.33 |
| teinase | | | | | | | | | | | |
| inhibitor 1 | | | | | | | | | | | |
| pigment | 49.98 | 6.14 | 0 | 3.07 | 5.35 | 4.81 | 4.07 | 3.96 | 3.37 | 0 | 2.87 |
| epithelium- | | | | | | | | | | | |
| derived | | | | | | | | | | | |
| factor | | | | | | | | | | | |
| isoform X1 | | | | | | | | | | | |
| acid | 28.16 | 1.56 | 0 | 0 | 0 | 3.52 | 1.4 | 4.03 | 0 | 0 | 8.71 |
| ceramidase | | | | | | | | | | | |
| isoform X1 | | | | | | | | | | | |
| coatomer | 13.68 | 0.982 | 0 | 0.724 | 1.59 | 0 | 0.515 | 0.542 | 0 | 0 | 0.914 |
| subunit | | | | | | | | | | | |
| beta | | | | | | | | | | | |
| isoform X1 | | | | | | | | | | | |
| 60S ribosomal | 96.38 | 0 | 0 | 0 | 0 | 0 | 0 | 0 | 0 | 0 | 0 |
| protein L10a | | | | | | | | | | | |
| isoform X1 | | | | | | | | | | | |
| cullin- | 44.48 | 1.71 | 0 | 0 | 1.8 | 0 | 0.939 | 0 | 0 | 0 | 0 |
| associated | | | | | | | | | | | |
| NEDD8- | | | | | | | | | | | |
| dissociated | | | | | | | | | | | |
| protein 1 | | | | | | | | | | | |
| alpha-L- | 29.17 | 0 | 0 | 0 | 0 | 0 | 0 | 0 | 0 | 0 | 0 |
| iduronidase | | | | | | | | | | | |
| isoform X1 | | | | | | | | | | | |
| torsin- | 19.23 | 1.86 | 0 | 0 | 2.39 | 5.08 | 1.75 | 1.47 | 2.15 | 0 | 2.21 |
| 1B-like | | | | | | | | | | | |
| isoform X1 | | | | | | | | | | | |

| | | | | | | | | | | |
|---|---|---|---|---|---|---|---|---|---|---|
| total HCP | 2866 | 1464 | 1586 | 2665 | 2829 | 2456 | 2298 | 2223 | 240 | 2762 |
| (relative ppm) | | | | | | | | | | |
| Pro A Yield (%) | 98.7 | 93.7 | 91.8 | 96.4 | 97.0 | 97.3 | 99.8 | 96.5 | 7.3 | 101.9 |
| Residual HCP by | 58 | 61 | 62 | 63 | 67 | 63 | 90 | 90 | 42 | 169 |
| ELISA (ng/mg) | | | | | | | | | | |

*EpiMatrix score expresses the T cell epitope concentration in a protein sequence.

### Example 2: Analysis of Sodium Caprylate Protein A Wash for Removing HCPs

To further analyze protein A wash conditions, wash buffers containing NaCl or sodium caprylate were compared as Protein A wash buffers to improve the overall clearance of host cell proteins (HCPs) in the process of dulaglutide purification. Specifically, clearing cathepsin D from the dulaglutide eluate is important because residual cathepsin D proteolytically cleaves dulaglutide, resulting in an undesirable des(1-22)/des(1-25) clipped form of dulaglutide. The protein A purification parameters for this wash study are described in Table 4 below, as well as the parameters for the dulaglutide manufacturing process.

TABLE 4
Dulaglutide protein A purification parameters

| Operating Parameters | Manufacturing Process | | Wash Study Conditions | |
|---|---|---|---|---|
| Protein A Resin | MabSelect SuRe LX | | MabSelect SuRe LX | |
| Load Temperature | 15-30° | C. | 15-30° | C. |
| Column Bed Height | 22-32.0 cm, 31.5 cm | | 10 cm pre-packed | |
| | Target | | HiScreen Column | |
| Column Diameter | 100 | cm | 0.77 | cm |
| Load Ratio | 10-18 mg DVI | | 15 mg DVI | |
| | Intermediate/mL resin | | Intermediate/mL resin | |
| Rinse Buffer [50 mM Tris, pH 8.0] | 2.2 CV (NLT 0.5) at 313 | | 2.2 CV at 0.78 mL/min ^{a} | |
| Column Volumes and Flow Rate | cm/hr (NMT 330) | | | |
| Sanitization [50 mM Sodium Hydroxide, 1M | 2 CV (NLT 1.8), 10-20 | | 2 CV, 10-20 min at 0.78 | |
| Sodium Chloride] | min at 313 cm/hr (NMT | | mL/min ^{a} | |
| Column Volumes, Contact Time and Flow | 330) | | | |
| Rate | | | | |
| Equilibration [50 mM Tris, pH 8.0] | ≥2 CVs at 300 cm/hr | | ≥2 CVs at 0.75 mL/min ^{b} | |
| Column Volumes and Flow Rate | (NMT 330) | | | |
| Load Flow Rate | 150 ± 15 | cm/hr | 0.37 | mL/min ^{c} |

| | | |
|---|---|---|
| Wash 1 [50 mM Tris, pH 8.0] | 5 CVs at 300 ± 30 cm/hr | 5 CVs at 0.75 mL/min ^{b} |
| Column Volumes and Flow Rate | | |
| Wash 2 Buffer | 50 mM Tris, 1M Sodium | TBD - See Table 5 |
| | Chloride, pH 8.0 | |
| Wash 2 Column Volumes and Flow Rate | 5 CVs at 300 ± 30 cm/hr | TBD CVs - See Table 2 at |
| | | 0.75 mL/min ^{b} |
| Wash 3 Buffer | 50 mM Tris, pH 8.0 | TBD - See Table 2 |
| Wash 3 Column Volumes and Flow Rate | 5 CVs at 300 ± 30 cm/hr | TBD CVs - See Table 2 at |
| | | 0.75 mL/min ^{b} |
| Wash 4 [50 mM Tris, pH 8.0] | N/A | 5 CVs at 0.75 mL/min ^{b} |
| Column Volumes and Flow Rate | | |
| Elution Peak Collection Criteria | Begin: ≥0.4 AU/mm | Begin: ≥0.4 AU/mm |
| AU per mm path length of UV flow cell or | End: 1.5 CV | End: 1.5 CV |
| Volume | | |
| Elution [10 mM Sodium Citrate, pH 3.0] | 5 CV (NLT 4.5) at 300 ± | 5 CV at 0.75 mL/min ^{b} |
| Column Volumes and Flow Rate | 30 cm/hr | |

^{a} Flowrate adjusted for a 6 minute residence time (based on target manufacturing bed height of 31.5 cm).
^{b} Flowrate adjusted for a 6.3 minute residence time (based on target manufacturing bed height of 31.5 cm).
^{c} Flowrate adjusted for a 12.6 minute residence time (based on target manufacturing bed height of 31.5 cm).

Several dulaglutide protein A purification runs were designed to test various washing conditions, such as serial washes and varying wash volumes, to find the optimal washing conditions for a sodium caprylate wash buffer in this step of dulaglutide purification, see Table 5 below.

CHO cells expressing dulaglutide were harvested and clarified by methods known in the art. The dulaglutide clarified cell broth was then loaded onto a chromatography column containing MabSelect SuRe LX protein A matrix, using the loading conditions in the Wash Study column in Table 4 above. The column was equilibrated with 50 mM Tris pH 8.0.

In the first set of protein A purification runs, wash buffers with serial concentrations of sodium caprylate were tested to determine the optimal strength (molarity range) of the sodium caprylate wash buffer needed for the wash, while minimizing the amount of caprylate added to the process, which would then potentially need to be cleared (see runs 3-6, Table 5). After the washes, dulaglutide was eluted according to the conditions listed in Table 4. The level of HCPs in the eluate was determined by HCP ELISA and LC-MS (as described in Example 1). The level of cathepsin D in the eluate was evaluated by a cathepsin D activity assay.

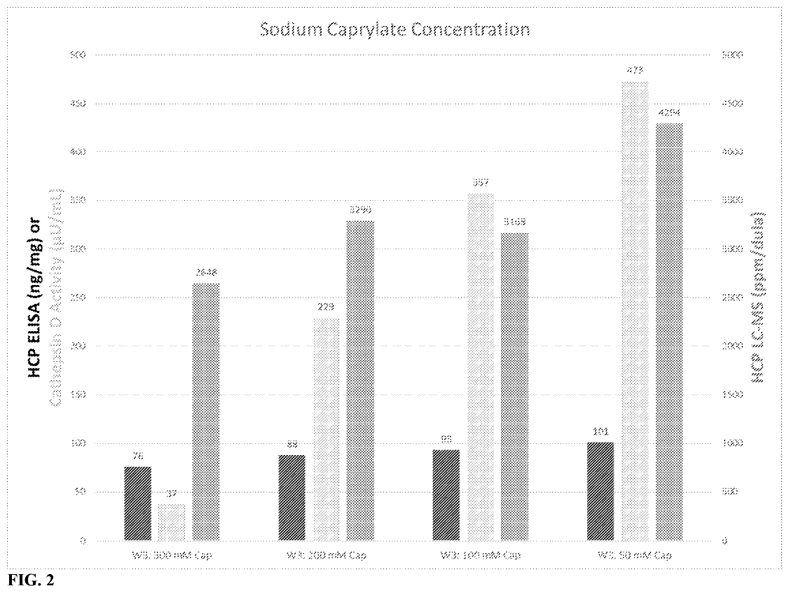

The results of this analysis show that a wash buffer containing 300 mM sodium caprylate significantly reduces cathepsin D activity in the eluate from protein A purification, while also minimizing levels of HCP (see FIG. 2), compared to a wash buffer with 1 M NaCl or wash buffers with lower concentrations of sodium caprylate.

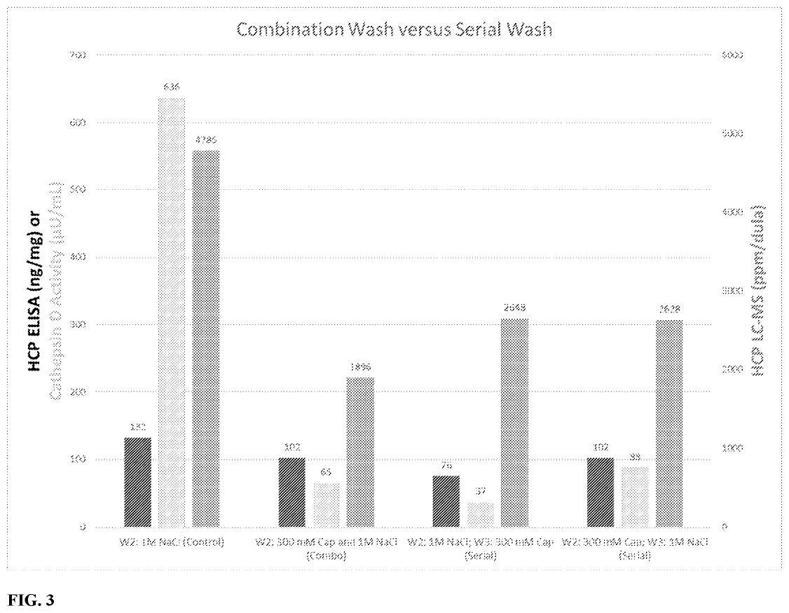

In a second set of protein A purification runs, washing the protein A chromatography matrix with a wash buffer containing a combination of NaCl/sodium caprylate was compared to serial washes with wash buffers containing either NaCl or sodium caprylate to determine orthogonality of washes, or benefit of serial washes versus a combination wash (see runs 7-9, Table 5). As shown in FIG. 3, a wash of sodium chloride followed in series by a wash with sodium caprylate results in the greatest reduction in cathepsin D activity and HCP levels in the eluate as assessed by ELISA, cathepsin D activity assay, and LC-MS.

In a third set of protein A purification runs, the impact of reducing the number of column volumes (CVs) of a wash buffer containing 300 mM sodium caprylate in the second wash step was determined. The protein A chromatography matrix was first equilibrated by rinsing with an equilibration buffer containing 50 mM Tris, pH 8.0, then washed with either a control condition: 5 CV of 50 mM Tris, pH 8.0, then 5 CV of 50 mM Tris, 1M NaCl, pH 8, then 5 CV of 50 mM Tris, pH 8.0; or the 5 CV wash, 3 CV wash or 2 CV wash condition: 5 CV of 50 mM Tris, pH 8.0, then X CV (5, 3, 2 CV) of 50 mM Tris, 1M NaCl, pH 8, then X CV of 50 mM Tris, 300 mM Sodium Caprylate, pH 8, then X CV 50 mM Tris, pH 8.0. The dulaglutide was eluted according to the conditions in Table 4 and the HCPs and cathepsin D were analyzed as described above.

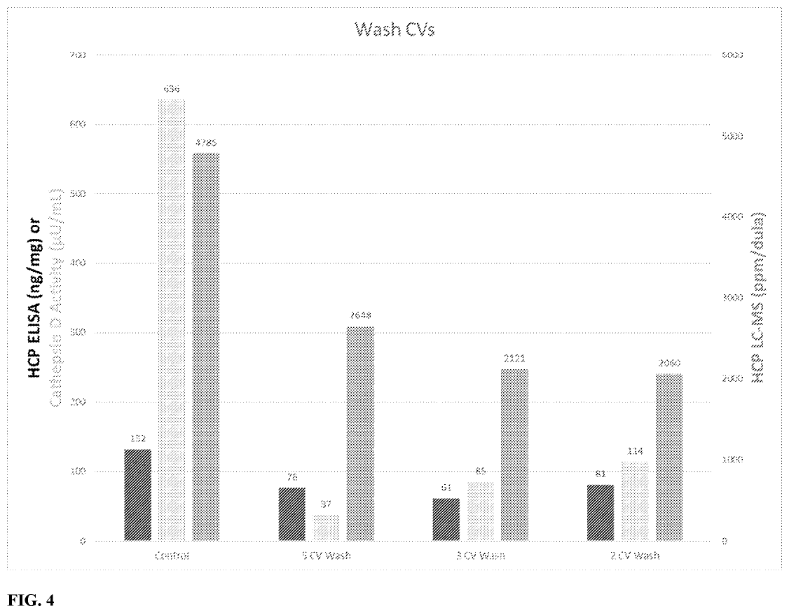

As shown in FIG. 4, the eluate from each run with a sodium caprylate wash buffer had significantly reduced HCPs and cathepsin D activity compared to the control wash buffer containing NaCl. Moreover, while two CVs resulted in a slight increase in cathepsin D activity compared to greater wash volumes (three or five CVs), HCP levels were comparable. In addition, five CVs of a wash buffer containing 300 mM sodium caprylate resulted in a 0.6% increase in aggregation of dulaglutide as compared to the control, while two or three CVs had no significant impact on aggregation (data not shown). Thus, using two CVs of sodium caprylate in the second wash step significantly reduces HCP levels and cathepsin D activity as compared to the control condition, while also reducing the total wash volume required and minimizing aggregation during the purification of dulaglutide.

Tables 5-7 below disclose the wash conditions that were tested in this study (Table 5), the % yield of dulaglutide after the protein A purification for each wash condition (Table 6), and the HCP levels and cathepsin D activity in the eluates for each wash condition (Table 7).

These results of the tested dulaglutide protein A purification conditions demonstrate optimal wash conditions for the protein A purification step of the dulaglutide manufacturing process to reduce the amount of residual HCPs. Specifically, these results demonstrate that a wash buffer containing 300 mM sodium caprylate used in a series of washes for in Protein A purification can effectively reduce HCP levels, and in particular the activity levels of cathepsin D, in the dulaglutide eluate, while also reducing the total wash volume required without increasing aggregation.

TABLE 5
Wash conditions for dulaglutide protein A purification

| Run | Wash #1 | Wash #2 | Wash #3 | Wash #4 |
|---|---|---|---|---|
| Run 13 | 50 mM Tris, pH 8 | 50 mM Tris, 1M NaCl, pH 8 | 50 mM Tris, pH 8 | None |
| (control) | | | | |
| Run 14 | 50 mM Tris, pH 8 | 50 mM Tris, 1M NaCl, pH 8 | 50 mM Tris, pH 8 | None |
| Run 15 | 50 mM Tris, pH 8 | 50 mM Tris, 300 mM Sodium | 50 mM Tris, pH 8 | None |
| | | Caprylate, pH 8 | | |
| Run 16 | 50 mM Tris, pH 8 | 50 mM Tris, 1M NaCl, pH 8 | 50 mM Tris, 300 mM Sodium | 50 mM Tris, pH 8 |
| | | | Caprylate, pH 8 | |
| Run 17 | 50 mM Tris, pH 8 | 50 mM Tris, 300 mM Sodium | 50 mM Tris, 1M NaCl, pH 8 | 50 mM Tris, pH 8 |
| | | Caprylate, pH 8 | | |
| Run 2 | 50 mM Tris, pH 8 | 50 mM Tris, pH 8 | 50 mM Tris, pH 8 | None |
| Run 3 | 50 mM Tris, pH 8 | 50 mM Tris, 300 mM Sodium | 50 mM Tris, pH 8 | None |
| | | Caprylate, pH 8 | | |
| Run 8 | 50 mM Tris, pH 8 | 50 mM Tris, 1M NaCl, pH 8 | 50 mM Tris, 300 mM Sodium | 50 mM Tris, pH 8 |
| | | | Caprylate, pH 8 | |
| Run 9 | 50 mM Tris, pH 8 | 50 mM Tris, 300 mM Sodium | 50 mM Tris, 1M NaCl, pH 8 | 50 mM Tris, pH 8 |
| | | Caprylate, pH 8 | | |
| Run 7 | 50 mM Tris, pH 8 | 50 mM Tris, 300 mM Sodium | 50 mM Tris, pH 8 | None |
| | | Caprylate, 1M NaCl, pH 8 | | |
| Run 26 | 50 mM Tris, pH 8 | 50 mM Tris, 1M NaCl, pH 8 | 20 mM Tris, pH 7 | None |
| Run 28 | 50 mM Tris, pH 8 | 50 mM Tris, 1M NaCl, pH 8 | 20 mM Tris, pH 8 | None |
| Run 27 | 50 mM Tris, pH 8 | 20 mM Tris, 1M NaCl, pH 7 | 20 mM Tris, pH 7 | None |
| Run 29 | 20 mM Tris, pH 7 | 20 mM Tris, 1M NaCl, pH 7 | 20 mM Tris, pH 7 | None |
| Run 30 | 20 mM Tris, pH 8 | 20 mM Tris, 1M NaCl, pH 8 | 20 mM Tris, pH 8 | None |
| Run 31 | 20 mM Tris, pH 7 | 20 mM Tris, pH 7 | 20 mM Tris, pH 7 | None |
| Run 13A | 50 mM Tris, pH 8 | 50 mM Tris, 1M NaCl, pH 8 | 50 mM Tris, pH 8 | None |
| Run 32 | 50 mM Tris, pH 8 | 50 mM Tris, 1M NaCl, pH 8 | 50 mM Tris, 200 mM Sodium | 50 mM Tris, pH 8 |
| | | | Caprylate, pH 8 | |
| Run 33 | 50 mM Tris, pH 8 | 50 mM Tris, 1M NaCl, pH 8 | 50 mM Tris, 100 mM Sodium | 50 mM Tris, pH 8 |
| | | | Caprylate, pH 8 | |
| Run 34 | 50 mM Tris, pH 8 | 50 mM Tris, 1M NaCl, pH 8 | 50 mM Tris, 50 mM Sodium | 50 mM Tris, pH 8 |
| | | | Caprylate, pH 8 | |
| Run 22 | 50 mM Tris, pH 8 | 50 mM Tris, 1M NaCl, pH 8 | 50 mM Tris, 300 mM Sodium | 50 mM Tris, pH 8 |
| | | | Caprylate, pH 8 | |
| Run 24 | 50 mM Tris, pH 8 | 50 mM Tris, 1M NaCl, pH 8 | 50 mM Tris, 50 mM Sodium | 50 mM Tris, pH 8 |
| | | | Caprylate, pH 8 | |
| Run 35 | 50 mM Tris, pH 8 | 50 mM Tris, 1M NaCl, pH 8 | 50 mM Tris, 300 mM Sodium | 50 mM Tris, pH 8 |
| | | | Caprylate, pH 8 | |
| Run 36 | 50 mM Tris, pH 8 | 50 mM Tris, 1M NaCl, pH 8 | 50 mM Tris, 50 mM Sodium | 50 mM Tris, pH 8 |
| | | | Caprylate, pH 8 | |
| Run 37 | 50 mM Tris, pH 8 | 50 mM Tris, 500 mM NaCl, | 50 mM Tris, pH 8 | None |
| | | pH 8 | | |
| Run 38 | 50 mM Tris, pH 8 | 50 mM Tris, 500 mM NaCl, | 50 mM Tris, 300 mM Sodium | 50 mM Tris, pH 8 |
| | | pH 8 | Caprylate, pH 8 | |
| Run 39 | 50 mM Tris, pH 8 | 50 mM Tris, 1.5M NaCl, pH 8 | 50 mM Tris, pH 8 | None |
| Run 40 | 50 mM Tris, pH 8 | 50 mM Tris, 1.5M NaCl, pH 8 | 50 mM Tris, 300 mM Sodium | 50 mM Tris, pH 8 |
| | | | Caprylate, pH 8 | |
| Run 41 | 50 mM Tris, pH 8 | 50 mM Tris, 1.5M NaCl, pH 8 | 50 mM Tris, 300 mM Sodium | 50 mM Tris, pH 8 |
| | | | Caprylate, pH 8 | |
| Run 42 | 100 mM Tris, pH 8 | 100 mM Tris, 1M NaCl, pH 8 | 100 mM Tris, pH 8 | None |
| Run 43 | 100 mM Tris, pH 8 | 100 mM Tris, 1M NaCl, pH 8 | 100 mM Tris, 300 mM Caprylate, | 100 mM Tris, pH 8 |
| | | | pH 8 | |
| Run 13B | 50 mM Tris, pH 8 | 50 mM Tris, 1M NaCl, pH 8 | 50 mM Tris, pH 8 | None |
| Run 44 | 10 mM Sodium Phosphate, 145 mM | 10 mM Sodium Phosphate, 1M | 10 mM Sodium Phosphate, 145 | None |
| | Sodium Chloride, pH 7.2 | Sodium Chloride, pH 7.2 | mM Sodium Chloride, pH 7.2 | |
| Run 45 | 10 mM Sodium Phosphate, 145 mM | 10 mM Sodium Phosphate, 1M | 10 mM Sodium Phosphate, pH 7.2 | None |
| | Sodium Chloride, pH 7.2 | Sodium Chloride, pH 7.2 | | |
| Run 46 | 10 mM Sodium Phosphate, 145 mM | 10 mM Sodium Phosphate, 1M | 10 mM Sodium Phosphate, 145 | None |
| | Sodium Chloride, pH 7.2 | Sodium Chloride, pH 7.2 | mM Sodium Chloride, pH 7.2 | |
| Run 47 | 10 mM Sodium Phosphate, 145 mM | 10 mM Sodium Phosphate, 1M | 10 mM Sodium Phosphate, pH 7.2 | None |
| | Sodium Chloride, pH 7.2 | Sodium Chloride, pH 7.2 | | |

TABLE 6
Processing data from the wash conditions in Table 5

| | 10 mM Sodium | 50 mM Tris, | Total | | | | | | | | |
|---|---|---|---|---|---|---|---|---|---|---|---|
| | Citrate, pH | 120 mM NaCl | Neut. | | | | | | | Avg. | |
| | 3.0 Added¹ | Added^{1, 2} | Buffer | | Cond. | Temp. | Conc. | | | Conc. | Yield |
| Run | mL | mL | mL | pH | ms/cm | ° C. | mg/mL | | | mg/mL | % |
| Run 13 | 2.82 | 8 | 10.82 | 8.12 | 6.915 | 21.2 | 3.52 | 3.51 | 3.56 | 3.53 | 89.2 |
| (control) | | | | | | | | | | | |
| Run 14 | 2.82 | 8 | 10.82 | 8.50 | 6.941 | 21.2 | 0.680 | 0.680 | 0.691 | 0.684 | 5.2 |
| Run 15 | 2.82 | 8 | 10.82 | 7.96 | 7.015 | 21.3 | 5.81 | 5.81 | 5.83 | 5.82 | 44.5 |
| Run 16 | 2.82 | 8 | 10.82 | 7.82 | 6.916 | 21.5 | 5.85 | 5.83 | 5.84 | 5.8 | 44.7 |
| Run 17 | 2.82 | 8 | 10.82 | 8.01 | 6.994 | 21.5 | 6.07 | 6.07 | 6.10 | 6.08 | 46.5 |
| Run 2 | 2.82 | 8 | 10.82 | 8.11 | 6.886 | 21.4 | 3.59 | 3.58 | 3.59 | 3.59 | 90.6 |
| Run 3 | 2.82 | 8 | 10.82 | 8.12 | 6.895 | 21.3 | 3.69 | 3.71 | 3.68 | 3.69 | 93.3 |
| Run 8 | 2.82 | 8 | 10.82 | 8.12 | 6.948 | 21.1 | 3.62 | 3.63 | 3.59 | 3.61 | 90.6 |
| Run 9 | 2.82 | 8 | 10.82 | 7.89 | 6.985 | 21.3 | 3.85 | 3.85 | 3.87 | 3.86 | 96.5 |
| Run 7 | 2.82 | 8 | 10.82 | 8.06 | 7.075 | 21.1 | 3.69 | 3.67 | 3.68 | 3.68 | 89.6 |
| Run 26 | 2.82 | 8 | 10.82 | 8.21 | 6.959 | 21.6 | 4.12 | 4.12 | 4.13 | 4.12 | 102.2 |
| Run 28 | 2.82 | 8 | 10.82 | 8.16 | 6.925 | 21.3 | 4.13 | 4.12 | 4.16 | 4.14 | 104.5 |
| Run 27 | 2.82 | 8 | 10.82 | 8.12 | 6.990 | 21.1 | 4.10 | 4.11 | 4.10 | 4.10 | 100.9 |
| Run 29 | 2.82 | 8 | 10.82 | 8.09 | 7.058 | 21.2 | 4.18 | 4.21 | 4.19 | 4.19 | 105.4 |
| Run 30 | 2.82 | 8 | 10.82 | 7.80 | 7.117 | 21.2 | 4.00 | 4.00 | 3.99 | 4.00 | 99.0 |
| Run 31 | 2.82 | 8 | 10.82 | 8.11 | 7.069 | 21.2 | 4.05 | 4.05 | 4.07 | 4.06 | 101.3 |
| Run 13A | 2.82 | 8 | 10.82 | 8.21 | 7.001 | 21.4 | 3.95 | 3.94 | 3.97 | 3.95 | 99.3 |
| Run 32 | 2.82 | 8 | 10.82 | 8.20 | 7.021 | 21.8 | 3.86 | 3.87 | 3.87 | 3.87 | 95.9 |
| Run 33 | 2.82 | 8 | 10.82 | 8.20 | 7.039 | 21.4 | 4.03 | 4.03 | 4.03 | 4.03 | 98.5 |
| Run 34 | 2.82 | 8 | 10.82 | 8.21 | 7.143 | 21.2 | 4.05 | 4.05 | 4.08 | 4.06 | 99.2 |
| Run 22 | 2.82 | 8 | 10.82 | 8.09 | 6.874 | 21.4 | 3.60 | 3.61 | 3.61 | 3.61 | 91.1 |
| Run 24 | 2.82 | 8 | 10.82 | 8.12 | 6.887 | 21.5 | 4.03 | 4.04 | 4.05 | 4.04 | 102.1 |
| Run 35 | 2.82 | 8 | 10.82 | 8.07 | 6.855 | 21.4 | 3.46 | 3.48 | 3.49 | 3.48 | 87.9 |
| Run 36 | 2.82 | 8 | 10.82 | 8.13 | 7.012 | 21.3 | 4.06 | 4.09 | 4.09 | 4.08 | 101.1 |
| Run 37 | 2.82 | 8 | 10.82 | 8.13 | 6.994 | 21.5 | 3.87 | 3.88 | 3.89 | 3.88 | 98.0 |
| Run 38 | 2.82 | 8 | 10.82 | 8.08 | 6.964 | 21.5 | 3.78 | 3.77 | 3.77 | 3.77 | 94.6 |
| Run 39 | 2.82 | 8 | 10.82 | 8.17 | 6.922 | 20.8 | 3.99 | 4.00 | 3.97 | 3.99 | 100.7 |
| Run 40 | 2.82 | 8 | 10.82 | 8.13 | 6.893 | 21.1 | 3.88 | 3.89 | 3.90 | 3.89 | 98.0 |
| Run 41 | 2.82 | 8 | 10.82 | 8.10 | 6.948 | 21.2 | 3.82 | 3.82 | 3.82 | 3.82 | 96.5 |
| Run 42 | 2.82 | 8 | 10.82 | 8.16 | 6.866 | 21.2 | 3.88 | 3.90 | 3.89 | 3.89 | 98.3 |
| Run 43 | 2.82 | 8 | 10.82 | 8.14 | 6.950 | 20.8 | 3.47 | 3.52 | 3.45 | 3.48 | 87.9 |
| Run 13B | 2.82 | 8 | 10.82 | 8.20 | 7.110 | 20.8 | 4.07 | 4.14 | 4.09 | 4.10 | 100.3 |
| Run 44 | 2.82 | 8 | 10.82 | 8.24 | 6.880 | 21.7 | 3.89 | 3.89 | 3.89 | 3.89 | 96.6 |
| Run 45 | 2.82 | 8 | 10.82 | 8.28 | 7.153 | 21.6 | 4.08 | 4.10 | 4.09 | 4.09 | 97.7 |
| Run 46 | 2.82 | 8 | 10.82 | 8.85 | 7.712 | 21.5 | 1.51 | 1.51 | 1.51 | 1.51 | 11.5 |
| Run 47 | 2.82 | 8 | 10.82 | 7.91 | 6.941 | 21.2 | 6.83 | 6.83 | 6.82 | 6.83 | 52.2 |

TABLE 7
Residual HCP data from the wash conditions in Table 5

| | | | | | | SEC- | | | | |
|---|---|---|---|---|---|---|---|---|---|---|
| | | Residual | Residual | Cathepsin | SEC- | HPLC | SEC- | Decrease | Decrease | Decrease in |
| | | HCP | HCP | D Activity | HPLC | Monomer | HPLC | in HCP | in HCP | Cathepsin D |
| | Yield | (ELISA) | (LC-MS) | Assay | Aggregates | Purity | Fragments | (ELISA)¹ | (LC- | Activity¹ |
| Run | % | ng/mg | ppm/Dula | mU/mL | % | % | % | % | MS)¹ | % |
| Run 13 | 89.2 | 250 | 3660.9 | 1,090 | 3.8 | 94.3 | 1.9 | 90% | −23% | 71% |
| (control) | | | | | | | | | | |
| Run 14 | 5.2 | 80 | 921.4 | 409 | 4.5 | 87.5 | 8.0 | −40% | −81% | −36% |
| Run 15 | 44.5 | 119 | 870.2 | 108 | 4.8 | 93.8 | 1.4 | −10% | −82% | −83% |
| Run 16 | 44.7 | 83 | 775.6 | 76 | 5.0 | 93.7 | 1.4 | −37% | −84% | −88% |
| Run 17 | 46.5 | 94 | 777.7 | 94 | 4.9 | 93.8 | 1.3 | −29% | −84% | −85% |
| Run 2 | 90.6 | 1762 | 5824.9 | 1,237 | 3.9 | 94.2 | 2.0 | 1235% | 22% | 94% |
| Run 3 | 93.3 | 99 | 2382.9 | 68 | 4.4 | 93.6 | 2.0 | −25% | −50% | −89% |
| Run 8 | 90.6 | 76 | 2647.9 | 37 | 4.4 | 93.6 | 2.0 | −43% | −45% | 94% |
| Run 9 | 96.5 | 102 | 2628.1 | 88 | 4.4 | 93.6 | 2.0 | −23% | −45% | −86% |
| Run 7 | 89.6 | 102 | 1896.4 | 65 | 4.6 | 93.4 | 1.9 | −23% | −60% | −90% |
| Run 26 | 102.2 | 125 | 5714.8 | 1019 | NT² | NT² | NT² | −5% | 19% | 60% |
| Run 28 | 104.5 | 134 | 5110.6 | 1189 | NT² | NT² | NT² | 2% | 7% | 87% |
| Run 27 | 100.9 | 130 | 5278.9 | 959 | NT² | NT² | NT² | −2% | 10% | 51% |
| Run 29 | 105.4 | 136 | 6867.4 | 1785 | NT² | NT² | NT² | 3% | 44% | 181% |
| Run 30 | 99.0 | 132 | 4315.7 | 1022 | NT² | NT² | NT² | 0% | −10% | 61% |
| Run 31 | 101.3 | 1866 | 6864.9 | 2401 | NT² | NT² | NT² | 1314% | 43% | 278% |
| Run 13A | 99.3 | 132 | 4785.0 | 636 | 3.7 | 94.4 | 2.0 | 0% | 0% | 0% |
| Run 32 | 95.9 | 88 | 3290.1 | 229 | 3.8 | 94.3 | 1.9 | −33% | −31% | −64% |
| Run 33 | 98.5 | 93 | 3167.6 | 357 | 3.8 | 94.4 | 1.8 | −30% | −34% | −44% |
| Run 34 | 99.2 | 101 | 4294.0 | 473 | 3.9 | 94.3 | 1.8 | −23% | −10% | −26% |
| Run 22 | 91.1 | 81 | 2060.1 | 114 | 3.4 | 94.6 | 2.0 | −39% | −57% | −82% |
| Run 24 | 102.1 | 115 | 3939.4 | 619 | 3.6 | 94.5 | 1.9 | −13% | −18% | −3% |
| Run 35 | 87.9 | 61 | 2121.1 | 85 | 3.8 | 94.2 | 2.1 | −54% | −56% | −87% |
| Run 36 | 101.1 | 109 | 3358.7 | 623 | 3.5 | 94.7 | 1.9 | −17% | −30% | −2% |
| Run 37 | 98.0 | 139 | 4376.5 | 681 | 3.5 | 94.6 | 1.9 | 5% | −9% | 7% |
| Run 38 | 94.6 | 71 | 2078.8 | 106 | 3.7 | 94.4 | 1.9 | −46% | −57% | −83% |
| Run 39 | 100.7 | 123 | 4462.7 | 785 | 3.4 | 94.7 | 1.9 | −7% | −7% | 23% |
| Run 40 | 98.0 | 66 | 2129.4 | 125 | 3.8 | 94.2 | 1.9 | −50% | −55% | −80% |
| Run 41 | 96.5 | 81 | 2043.0 | 153 | NT² | NT² | NT² | −39% | −57% | −76% |
| Run 42 | 98.3 | 107 | 2825.8 | 511 | 3.4 | 94.8 | 1.9 | −19% | −41% | 20% |
| Run 43 | 87.9 | 50 | 1345.6 | 75 | 4.2 | 93.8 | 2.0 | −62% | −72% | −88% |
| Run 13B | 100.3 | 111 | 3967.8 | 837 | 3.5 | 94.6 | 1.9 | −16% | −17% | 32% |
| Run 44 | 96.6 | 114 | 3302.7 | 641 | 3.2 | 95.0 | 1.7 | −14% | −31% | 1% |
| Run 45 | 97.7 | 109 | 3819.6 | 762 | 2.9 | 95.4 | 1.7 | −17% | −20% | 20% |
| Run 46 | 11.5 | 33 | 1329.7 | 437 | 4.5 | 91.6 | 3.8 | −75% | −72% | −31% |
| Run 47 | 52.2 | 96 | 1282.0 | 629 | 3.9 | 94.8 | 1.2 | −27% | −73% | −1% |

¹% Decrease from current process condition/control (50 mM Tris, 1M Sodium Chloride, pH 8.0 - Run #13A Condition).
²NT = not tested.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Other embodiments are within the following claims.

1. A method of purifying dulaglutide from a mixture comprising the dulaglutide and one or more host cell protein (HCP), the method comprising the steps of:
(a) applying the mixture to a chromatography column comprising a Protein A chromatography matrix, under conditions whereby the dulaglutide binds to the chromatography matrix; and
(b) washing the chromatography matrix with a sodium caprylate wash buffer comprising 150-500 mM sodium caprylate.

2. The method of claim 1, wherein the sodium caprylate wash buffer comprises 150-350 mM sodium caprylate.

3. The method of claim 1, wherein the sodium caprylate wash buffer comprises 200-350 mM sodium caprylate.

4. The method of claim 1, wherein the sodium caprylate wash buffer comprises about 300 mM sodium caprylate.

5. The method of claim 1, wherein the sodium caprylate wash buffer comprises about 50 mM Tris.

6. The method of claim 1, wherein the sodium caprylate wash buffer comprises 0.5-1.5 M NaCl.

7. The method of claim 1, wherein the sodium caprylate wash buffer comprises about 1M NaCl.

8. The method claim 1, wherein the sodium caprylate wash buffer has a pH of 7-9.

9. The method of claim 1, wherein the sodium caprylate wash buffer has a pH of about 8.

10. The method of claim 1, further comprising washing the chromatography matrix with a NaCl wash buffer comprising 0.5-1.5 M NaCl.

11. The method of claim 10, wherein the NaCl wash buffer comprises about 1M NaCl.

12. The method of claim 10, wherein the NaCl wash buffer comprises about 50 mM Tris.

13. The method of claim 10, wherein the NaCl wash buffer has a pH of 7-9.

14. The method of claim 10, wherein the NaCl wash buffer has a pH of about 8.

15. The method of claim 10, wherein the chromatography column is washed with the NaCl wash buffer prior to being washed with the sodium caprylate wash buffer.

16. The method of claim 10, wherein the chromatography column is washed with the sodium caprylate wash buffer prior to being washed with the NaCl wash buffer.

17. The method of claim 1, wherein the chromatography matrix is equilibrated with a Tris buffer at a pH of 7-9 between step (a) and step (b).

18. The method of claim 17, wherein the Tris buffer comprises 10-100 mM Tris.

19. The method of claim 17, wherein the Tris buffer comprises about 50 mM Tris.

20. The method of claim 17, wherein the Tris buffer has a pH of about 8.0.

21. The method of claim 1 any one of the preceding claims, wherein the chromatography matrix is washed with 2-10 column volumes of the sodium caprylate wash buffer.

22. The method of claim 21, wherein the chromatography matrix is washed with about 2 column volumes of the sodium caprylate wash buffer.

23. The method of claim 10, wherein the chromatography matrix is washed with 2-10 column volumes of the NaCl wash buffer.

24. The method of claim 23, wherein the chromatography matrix is washed with about 2 or about 5 column volumes of the NaCl wash buffer.

25. The method of claim 1, wherein the chromatography matrix is sequentially washed with:
(a) about 2 column volumes of a NaCl wash buffer comprising about 50 mM Tris and about 1 M NaCl, with a pH of about 8; and
(b) about 2 column volumes of a sodium caprylate wash buffer comprising about 50 mM Tris and about 300 mM sodium caprylate, with a pH of about 8.

26. The method of claim 25, wherein the chromatography matrix is washed with the NaCl wash buffer at a flow rate of 300±30 cm/hr.

27. The method of claim 25, wherein the chromatography matrix is washed with the sodium caprylate wash buffer at a flow rate of 300±30 cm/hr.

28. The method of claim 25, further comprising washing the chromatography matrix with a Tris buffer comprising about 50 mM Tris at a pH of about 8.0.

29. The method of claim 1, further comprising contacting the chromatography matrix with an elution buffer to produce an eluate containing dulaglutide.

30. The method of claim 29, wherein the elution buffer has a pH of 2.5-4.

31. The method of claim 29, wherein the elution buffer has a pH of about 3.

32. The method of claim 29, wherein the elution buffer comprises sodium citrate.

33. The method of claim 29, wherein the elution buffer comprises 5-25 mM sodium citrate.

34. The method of claim 29, wherein the elution buffer comprises about 10 mM sodium citrate.

35. The method of claim 29, wherein the eluate is monitored by absorbance using a spectrophotometer.

36. The method of claim 29, wherein the eluate comprises at least 70% of the dulaglutide present in the mixture.

37. The method of claim 1, wherein the chromatography column has a diameter of 75-150 cm.

38. The method of claim 1, wherein the chromatography column has a diameter of about 100 cm.

39. The method of claim 1, wherein the Protein A chromatography matrix has an average particle size of 80-90 μm.

40. The method of claim 1, wherein the Protein A chromatography matrix has an average particle size of about 85 μm.

41. The method of claim 1, wherein the Protein A chromatography matrix has an average particle size of about 60 μm.

42. The method of claim 1, wherein the protein A chromatography matrix is stable in alkali.

43. The method of claim 1, wherein the one or more HCP is a protease selected from the group consisting of serine protease, aspartic protease, cysteine protease, metalloprotease, and aminopeptidase, or a combination thereof.

44. The method of claim 1, wherein the one or more HCP is cathepsin D.

45. The method of claim 29, wherein the eluate comprises a reduced amount of the one or more HCP compared to the mixture.

46. The method of claim 29, wherein the eluate comprises no more than 100 ng/mg of HCPs.

47. The method of claim 45, wherein the amount of HCPs is measured by mass spectrometry or ELISA.

48. The method of claim 29, wherein the eluate comprises a reduced amount of cathepsin D compared to the mixture.

49. The method of claim 29, wherein the eluate comprises no more than 100 ng/mg cathepsin D.

50. The method of claim 48, wherein the amount of cathepsin D is measured by mass spectrometry or ELISA.

51. The method of claim 48, wherein the amount of cathepsin D is measured by an enzymatic assay.

52. The method of claim 47, wherein the mass spectrometry is LC-MS.

53. The method of claim 29, wherein the eluate comprises less than 300 μU/mL cathepsin D activity.

54. Dulaglutide produced by the method of claim 1.

55. A composition comprising dulaglutide produced by the method of claim 1.

56. A method of purifying dulaglutide from a mixture comprising the dulaglutide and one or more host cell protein (HCP), the method comprising the steps of:
(a) applying the mixture to a chromatography column comprising a Protein A chromatography matrix, under conditions whereby the dulaglutide binds to the chromatography matrix;
(b) washing the chromatography matrix with about 2 column volumes of a NaCl wash buffer comprising about 50 mM Tris and about 1 M NaCl, with a pH of about 8;
(c) washing the chromatography matrix with about 2 column volumes of a sodium caprylate wash buffer comprising about 50 mM Tris and about 300 mM sodium caprylate, with a pH of about 8;
(d) washing the chromatography matrix with a Tris buffer comprising about 50 mM Tris at a pH of about 8.0; and
(e) contacting the chromatography matrix with an elution buffer comprising about 10 mM sodium citrate and having a pH of about 3 to produce an eluate containing dulaglutide;
wherein the eluate comprises: at least 70% of the dulaglutide present in the mixture and no more than 100 ng/mg of HCPs; and wherein the one or more HCPs includes cathepsin D.


SEQUENCE LISTING (4 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, synthetic construct
HGEGTFTSDVSSYLEEQAAKEFIAWLVKGGG

SEQ ID NO: 3 - protein, 5 aa, synthetic construct
GGGGS

SEQ ID NO: 4 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG